(19) United States
(12) Patent Application Publication
(10) Pub. No.: US 2011/0281356 A1
(43) Pub. Date: Nov. 17, 2011

(54) COMPOUNDS FOR STEM CELL DIFFERENTIATION

(75) Inventors: Mark Mercola, La Jolla, CA (US); John Cashman, San Diego, CA (US); Marion Lanier, San Diego, CA (US); Erik Willems, San Diego, CA (US)

(73) Assignees: Human BioMolecular Research Institute, San Diego, CA (US); Sanford-Burnham Medical Research Institute, La Jolla, CA (US)

(21) Appl. No.: 13/107,592

(22) Filed: May 13, 2011

Related U.S. Application Data
(63) Continuation-in-part of application No. 12/561,235, filed on Sep. 16, 2009.
(60) Provisional application No. 61/097,823, filed on Sep. 17, 2008.

(51) Int. Cl. C12N 5/0735; C12N 5/074; C07D 215/54

(52) U.S. Cl. 435/377; 546/165

(57) ABSTRACT

Methods and small molecule compounds for stem cell differentiation are provided. One example of a class of compounds that may be used is represented by the compound of Formula I:

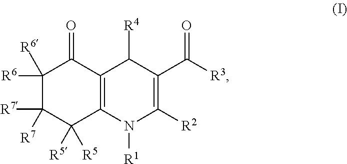

or a pharmaceutically acceptable salt or solvate thereof, wherein R¹, R², R³, R⁴, R⁵, R^{5′}, R⁶, R^{6′}, R⁷, R^{7′} are as described herein.

### CROSS REFERENCE TO RELATED APPLICATION(S)

This application is a continuation-in-part of U.S. patent application Ser. No. 12/561,235 filed Sep. 16, 2009, which claims the benefit under 35 USC §119(e) of U.S. Provisional Patent Application Ser. No. 61/097,823, filed Sep. 17, 2008, the entire content of each is hereby incorporated by reference.

### GRANT INFORMATION

This invention is made with government support under Comprehensive NIH Grant No. HL071913 awarded by the National Institutes of Health. The Government has certain rights in the invention.

### FIELD OF THE INVENTION

The disclosure relates generally to small molecule compounds and more specifically to derivatives of dihydropyridines, benzimidazoles, phenothiazines, and tamoxifen, and their use in stem cell differentiation.

### BACKGROUND OF THE INVENTION

Stem cells are a type cells that could be a source for the replacement of damaged or diseased tissues, and embryonic stem cells (ESCs) are a type of stem cells attracting particular interest. It has been previously shown that embryonic stem cells have the capacity to differentiate into many different cell types including heart, bone, neurons, liver tissue, and the like, both in vitro and in vivo. The differentiation potential of these cells has created substantial interest, since embryonic stem cells can thus provide a resource for replacing diseased cells for regenerating purposes.

ESCs are pluripotent cells which are derived from the inner cell mass of a blastocyst. The unique characteristics of ESCs are their capacities to regenerate themselves and to be capable of developing into various cell types of all three embryonic germ layers, ectoderm, mesoderm and endoderm, under appropriate environments. Such differentiated cell types include, but are not limited to, muscle, nerve, heart, liver, bone and blood. The potential of ESCs, induced pluripotent stem cells (iPSCs), adult or tissue specific stem cells and the like to grow into specialized cells attracts interest for research and disease treatment using these cells. The clinical application of stem cells involves harvest of the cells and transplantation of cells into failing organs to restore the function of the organs with or without prior in vitro differentiation.

Adult cardiomyocytes retain little, if any, ability to replicate, thus, heart failure is principally a disease of cardiomyocyte loss. No stem cell therapies to date have yielded significant replacement. Rather, transplanted cells, if they persist, produce endothelial cells or fibroblasts, and their reported ameliorating effects on heart function are probably the consequence of improvements in contractility, perfusion or other impaired processes. Replacement strategies by transplantation or stimulation of endogenous regeneration have been hypothesized. Whether endogenous cardiomyocyte stem cells exist and can be mobilized remains controversial, although a few populations have been proposed. Cardiomyocytes have potential in restoring heart function after myocardial infarction or in heart failure. Human embryonic stem cells (hESCs) are a potential source of transplantable cardiomyocytes but detailed comparison of hESC-derived cardiomyocytes with primary human cardiomyocytes is necessary before transplantation into patients becomes feasible.

While a clear alternative is to use hESCs, their cardiomyocyte yields are currently low. Generating sufficient new myocytes is a major obstacle when 25% of the ˜4 billion cardiomyocytes in the average left ventricle are lost in infarction-induced heart failure. Transplanted cell survival is currently about 5%, thus improving replication of committed precursors either pre- or post-implantation is essential. Interestingly, transplanted hESC-derived cardiomyocytes tend to retain some proliferative capacity, perhaps due to their relative immaturity; however, the number of engrafted cells remains small in all studies to date, thereby reinforcing the need for molecules that promote cell division.

The American Diabetes Association estimates that there are currently 5 million people in the United States with confirmed diabetes, and over 10 million at risk. The cost of this disease and its sequelae to the American economy is staggering. Care of diabetics consumes a total of $98 billion per year, accounting for one of every seven healthcare dollars spent in the U.S. There are 24,000 new cases of diabetes-caused blindness caused by diabetes each year. Diabetes is the leading cause of kidney failure, contributing about 40% of new dialysis patients. Diabetes is also the most frequent cause of lower limb amputation, with 56,000 limbs lost to diabetes each year. The per capita health care costs incurred per diabetic person is $10,071 annually, compared with $2,669 for non-diabetics.

Type I diabetes mellitus (also known as insulin-dependent diabetes) is a severe condition accounting for 5-10% all diabetics. The pathology arises because the patient's insulin-secreting beta cells in the pancreas have been eliminated by an autoimmune reaction. Under current practice, the condition is managed by regular injection of insulin, constant attention to diet, and continuous monitoring of blood glucose levels to adjust the insulin dosing. It is estimated that the market for recombinant insulin will reach $4 billion by 2005. Of course, the availability of insulin is life-saving for Type I diabetics. But there is no question that the daily regimen of administration and monitoring that diabetics must adhere to is troublesome to the end user, and not universally effective.

Developmental work has been done in several institutions to capitalize on the promise of pluripotent stem cells from the embryo to differentiate into other cell types. Cells bearing features of the islet cell lineage have reportedly been derived from embryonic cells of the mouse. Thus, it is necessary to develop new paradigms to differentiate human pluripotent cells into fully functional differentiated cell types.

### SUMMARY OF THE INVENTION

The present disclosure addresses these needs and more by providing new compounds, compositions and methods for differentiating human pluripotent cells into fully functional differentiated cell types.

In one embodiment the disclosure provides a compound of Formula I:

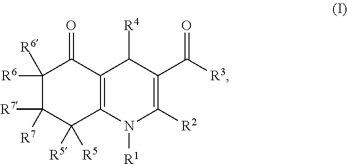

or a pharmaceutically acceptable salt or solvate thereof, wherein:

R¹ is independently hydrogen, (C₁-C₆)alkyl or a moiety forming a salt;

R² is independently hydrogen, (C₁-C₆)alkyl, CF₃ or C₂F₅;

R³ is independently OR⁸ or NR⁸R^{8′};

R⁴ is independently substituted or unsubstituted phenyl, substituted or unsubstituted pyridine, wherein phenyl or pyridine is optionally independently substituted with 1 to 3 R⁹ substituents;

R⁵, R^{5′}, R⁶, R^{6′}, R⁷, and R^{7′} are each independently hydrogen or (C₁-C₆)alkyl, phenyl, heteroaryl;

R⁸ and R^{8′} are each independently hydrogen, (C₁-C₆)alkyl, (C₃-C₈)cycloalkyl, substituted or unsubstituted heterocyclyl, aryl, (C₁-C₆)alkylaryl, or (C₁-C₆)alkylNR¹⁰R^{10′};

each R⁹ is independently hydrogen, halogen, (C₁-C₆)alkyl, (C₁-C₆)alkoxy, cyclo(C₁-C₆)alkyl, substituted or unsubstituted phenyl, substituted or unsubstituted pyridine, substituted or unsubstituted indolyl; substituted or unsubstituted pyrrolidinyl, or substituted or unsubstituted piperidinyl, wherein phenyl, pyridine, indolyl, pyrrolidinyl and piperidinyl are each optionally independently substituted with hydrogen, halogen, or (C₁-C₆)alkyl; and

R¹⁰ and R^{10′} are each independently hydrogen, (C₁-C₆)alkyl, aryl, or (C₁-C₆)alkylaryl.

In another aspect the disclosure provides methods for producing differentiated cells from stem cells by contacting the stem cells with a compound of Formula I.

In another aspect the disclosure provides dihydropyridine-based compounds of structure IA or IB in the form of a free base or a pharmaceutically acceptable salt, hydrate, solvate or N-oxide thereof:

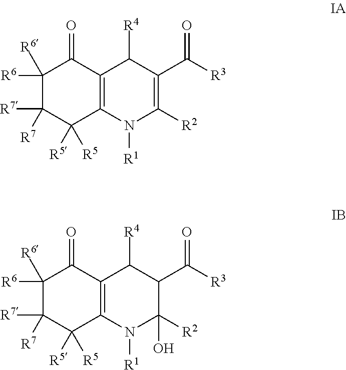

wherein R₁ is independently hydrogen, (C₁-C₆)alkyl, or is a moiety forming a salt; R₂ is independently hydrogen, (C₁-C₆)alkyl, aryl, or heteroaryl; R_{2′} is independently hydrogen, (C₁-C₆)alkyl, CF₃ or C₂F₅; R₃ is independently hydrogen, (C₁-C₆)alkyl optionally substituted by an amine, aryl, 2-tetrahydrofurylmethyl, an aliphatic tertiary amine, 4-methoxybenzyl, OR⁸ or NR⁸R^{8′}, or R₂ and R₃ may be joined together to form a 5 or 6 member ring lactone; R₄ is independently hydrogen, (C₁-C₆)alkyl, a 2- or 4-R₅-substituted aromatic ring selected from a phenyl, pyridyl, aryl, and heteroaryl; R⁸ and R^{8′} are each independently hydrogen, (C₁-C₆)alkyl, (C₃-C₈)cycloalkyl, substituted or unsubstituted heterocyclyl, aryl, (C₁-C₆)alkylaryl, or (C₁-C₆)alkylNR¹⁰R^{10′}; R¹⁰ and R^{10′} are each independently hydrogen, (C₁-C₆)alkyl, aryl, or (C₁-C₆)alkylaryl; and R₅, R_{5′}, R₆, R_{6′}, R₇, R_{7′} are each independently hydrogen, (C₁-C₆)alkyl, aryl, optionally substituted phenyl, heteroaryl, a heterocyclic ring, an aliphatic tertiary amine, or halogen.

In another aspect the disclosure provides methods for stem cell differentiation, comprising contacting the embryonic stem cells with a dihydropyridine-based compound of structure IA or IB in the form of a free base or a pharmaceutically acceptable salt, hydrate, solvate or N-oxide thereof, wherein R₁, R₂, R_{2′}, R₃, R₄, R₅, R_{5′}, R₆, R_{6′}, R₇, and R_{7′} are as described above.

In another aspect the disclosure provides benzimidazole-based compounds of structure II in the form of a free base or a pharmaceutically acceptable salt, hydrate, solvate or N-oxide thereof:

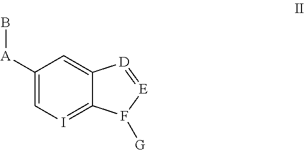

wherein A is independently a bond, NH, O, CH₂, C(O)NH, C(O)O, or CH₂NH; B is independently an aromatic ring or a saturated 4, 5, or 7 membered ring optionally containing a heteroatom such as N or O, directly attached to A, or with a saturated spacer such as a methylene of a 4-methylpiperidine; D is independently N or CH, E is independently N, CH, C═O, C—R, C—NH₂, or C—N(R)₂; F is independently N or CH; G is independently phenyl, pyridine or cyclohexyl optionally substituted by 1 to 5 R₁; I is independently N or CH; R₁ is independently hydrogen or (C₁-C₆)alkyl; R₂ is independently hydrogen, (C₁-C₆)alkyl, (C₁-C₆)alkoxy, (C₁-C₆)thioalkoxy, hydroxy, halogen, haloalkyl, CF₃, or C₂F₅; R is independently hydrogen or (C₁-C₆)alkyl.

In another aspect the disclosure provides methods for stem cell differentiation, comprising contacting the embryonic stem cells with a benzimidazole-based compound of structure I in the form of a free base or a pharmaceutically acceptable salt, hydrate, solvate or N-oxide thereof, wherein A, B, D, E, F, G, and I are as described above.

In another aspect the disclosure provides benzimidazole-based compounds of structure II, IIA, IIB, IIC, IID, IIE, IIF, IIG, IIH, IIJ, IIK, IIL, and IIM in the form of a free base or a pharmaceutically acceptable salt, hydrate, solvate or N-oxide thereof:

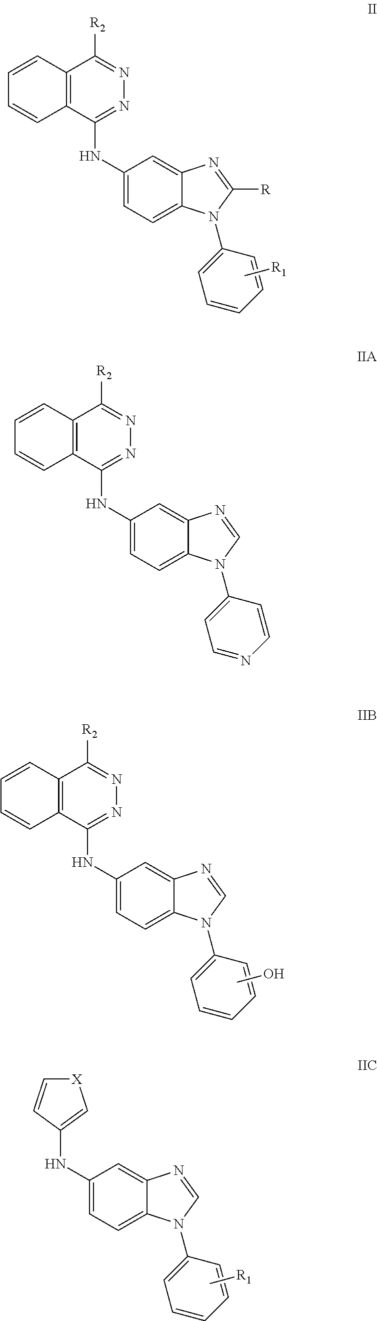

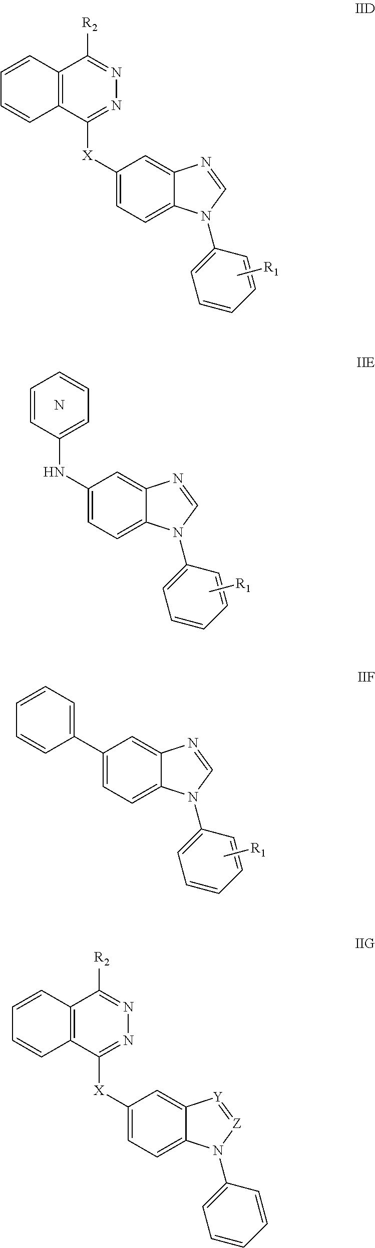

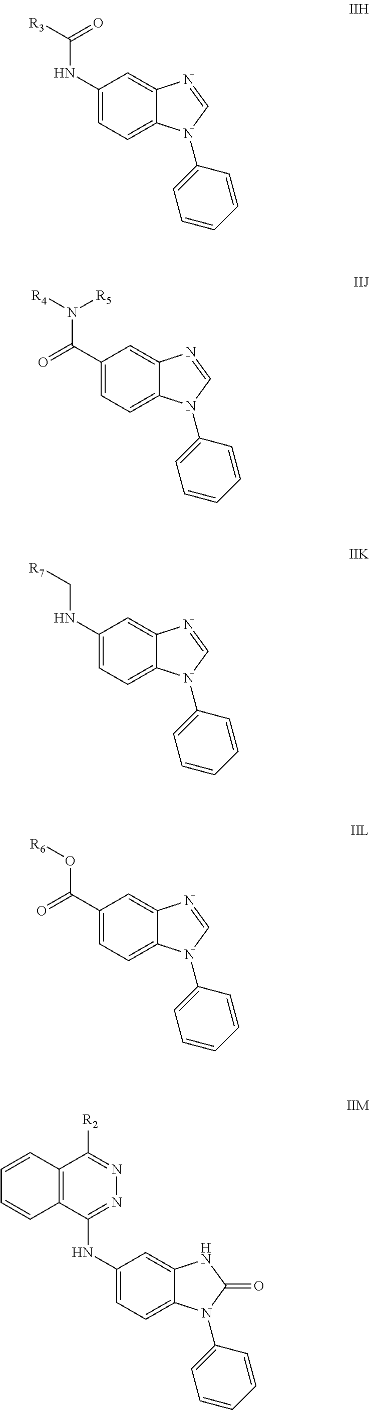

wherein R is independently hydrogen, methyl, or amino; R₁ is independently hydrogen, hydroxyl, methyl, trifluoromethyl, methoxy or methylthio; R₂ is independently hydrogen, phenyl, benzyl, methoxy, methyl or halogen; R₃, R₄, R₅, R₆, R₇ are C₁-C₆ alkyl, optionally substituted phenyls or heteroaryls, each of Y and Z is independently CH or N; and X is independently CH₂, NH, O, S, S═O, SO₂, CH(OH), or C═O.

In another aspect the disclosure provides methods for stem cell differentiation, comprising contacting the embryonic stem cells with a benzimidazole-based compound of structure II, IIA, IIB, IIC, IID, IIE, IIF, IIG, IIH, IIJ, IIK, IIL, or IIM as described above, in the form of a free base or a pharmaceutically acceptable salt, hydrate, solvate or N-oxide thereof.

In another aspect the disclosure provides phenothiazine-based compounds of structures III and IV in the form of a free base or a pharmaceutically acceptable salt, hydrate, solvate or N-oxide thereof:

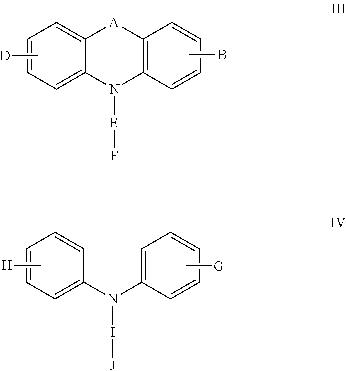

wherein A is independently S, O or (CH₂)ₙ, and n is independently an integer from 0 to 2; B and D are each independently alkyl, aryl, halo, SCH₃, or CF₃; E is independently (CH₂)ₘ, and m is independently an integer from 0 to 5; F is independently a primary or secondary alkyl amine, cyclic amine, an aryl amine or aliphatic cyclic amine, G and H are each independently alkyl, aryl, halo, SCH₃, or CF₃; I is independently (CH₂)ₘ, and m is independently an integer from 0 to 5; J is independently a primary or secondary alkyl amine, cyclic amine, an aryl amine or aliphatic cyclic amine.

In another aspect the disclosure provides phenothiazine-based compounds of structure IIIA (10-(aminoalkyl)-2-(substituted)-10H-phenothiazine):

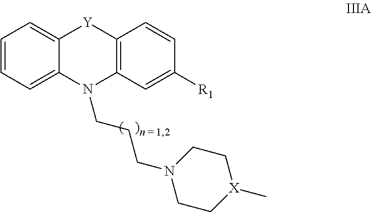

wherein R₁ is independently hydrogen, CH₃, SCH₃, or CF₃; X is independently CH, N, or O; Y is independently S, O, (CH₂)ₙ, where n is an integer from 0 to 2.

In another aspect the disclosure provides methods for stem cell differentiation, comprising contacting embryonic stem cells with a phenothiazine-based compound of structure III, IV, or IIIA in the form of a free base or a pharmaceutically acceptable salt, hydrate, solvate or N-oxide thereof:

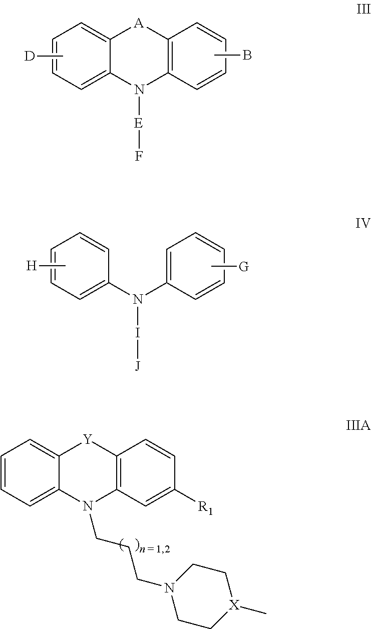

wherein A, B, D, E, F, G, H, I, J, X, Y, and R₁ are as described above.

In another aspect the disclosure provides tamoxifen-based compounds of structure V, VI or VII in the form of free base or a pharmaceutically acceptable salt, hydrate, solvate or N-oxide thereof:

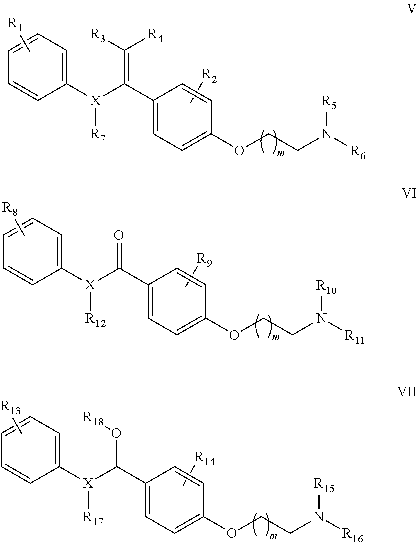

wherein for compound V, X is independently a bond, CH₂, or CHR₇; R₁ is independently methyl, ethyl, (C₁-C₆)alkyl, halogen, phenyl, methoxy, phenoxy, nitro, trifluoromethyl, or alkylamino; R₂ is independently methyl, ethyl, phenyl, (C₁-C₆)alkyl, trifluoromethyl, or halogen; R₃ is independently methyl, ethyl, (C₁-C₆)alkyl, phenyl, or aryl; R₄ is independently methyl, ethyl, (C₁-C₆)alkyl, phenyl, or aryl; R₅ is independently methyl, ethyl, (C₁-C₆)alkyl, phenyl, or aryl; R₆ is independently methyl, ethyl, (C₁-C₆)alkyl, phenyl, or aryl, wherein R₅ and R₆ may be joined via a ring, R₇ is independently methyl, ethyl, propyl, (C₁-C₆)alkyl, phenyl, or benzyl; and m is independently 0-4 methylene units; and wherein for compound VI, X is independently a bond, CH₂, or CHR₁₂; R₈ is independently methyl, ethyl, (C₁-C₆)alkyl, halogen, phenyl, methoxy, phenoxy, nitro, trifluoromethyl, or alkylamino, R₉ is independently methyl, ethyl, phenyl, (C₁-C₆)alkyl, trifluoromethyl, or halogen; R₁₀ is independently methyl, ethyl, (C₁-C₆)alkyl, phenyl, or aryl, R₁₁ is independently methyl, ethyl, (C₁-C₆)alkyl, phenyl, or aryl, wherein R₁₀ and R₁₁ can be joined via a ring; R₁₂ is independently methyl, ethyl, propyl, (C₁-C₆)alkyl, phenyl, or benzyl; and m is independently 0-4 methylene units; and wherein for compound VII, X is independently a bond, CH₂, or CHR₁₇; R₁₃ is independently methyl, ethyl, (C₁-C₆)alkyl, halogen, phenyl, methoxy, phenoxy, nitro, trifluoromethyl, or alkylamino; R₁₄ is independently methyl, ethyl, phenyl, (C₁-C₆)alkyl, trifluoromethyl, or halogen; R₁₅ is independently methyl, ethyl, (C₁-C₆)alkyl, phenyl, or aryl; R₁₆ is independently methyl, ethyl, (C₁-C₆)alkyl, phenyl, or aryl, wherein R₁₅ and R₁₆ may be joined via a ring; R₁₇ is independently methyl, ethyl, propyl, (C₁-C₆)alkyl, phenyl, or benzyl; R₁₈ is independently methyl, ethyl, propyl, (C₁-C₆)alkyl, phenyl, or benzyl; and m is independently 0-4 methylene units.

In another aspect the disclosure provides methods for stem cell differentiation, comprising contacting the embryonic stem cells with a compound of structure V, VI or VII, in the form of free base or a pharmaceutically acceptable salt, hydrate, solvate or N-oxide thereof.

### BRIEF DESCRIPTION OF THE DRAWINGS

FIG. 1 demonstrates schematically comparison of heart induction in mouse embryos and mESCs.

FIG. 2 provides a summary model for signaling pathways in cardiomyocyte formation.

FIG. 3 provides data on windows of exposure and activity for some of the disclosed compounds.

FIG. 4 provides a diagram of the serum-free assay used for the biological MOA studies.

FIG. 5 demonstrates synergy of the small molecules with activin/Nodal signaling.

FIG. 6 provides a diagram of step in stem cell cardiogenesis when gene expression data indicate that the compounds act.

FIG. 7 is a graph showing the effects of Wnt in combination with some of the disclosed compounds.

### DETAILED DESCRIPTION OF THE INVENTION

The following terms, definitions and abbreviations apply. Abbreviations used herein have their conventional meaning within the chemical and biological arts.

The term “lipophilic” refers to moieties having an affinity for lipids and other fat-like substances, tending to combine with, and capable of dissolving, them.

The term “cardiomyocytes” refers to cells of muscular tissue in the heart.

The term “embryonic stem cell” refers to cell from the inner group of cells of an early embryo (blastocyst), with the potential to become most or all of the body tissues.

The term “stem cell differentiation” refers to series of events involved in the development of specialized cells from stem cells, where the specialized cells have specific structural, functional, and biochemical properties.

The term “patient” refers to organisms to be treated by the methods of the disclosure. Such organisms include, but are not limited to, humans. In the context of the disclosure, the term “subject” generally refers to an individual who will receive or who has received treatment described below (e.g., administration of the compounds of the disclosure, and optionally one or more additional therapeutic agents).

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched chain, or cyclic hydrocarbon radical, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂CCCH₂—, —CH₂CH₂CH(CH₂CH₂ CH₃)CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—CH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings, which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g., “3 to 7 membered”), the term “member” referrers to a carbon or heteroatom.

The term “oxo” as used herein means an oxygen that is double bonded to a carbon atom.

The terms “heterocycle” and “heterocyclic” refer to a monovalent unsaturated group having a single ring or multiple condensed rings, from 1 to 8 carbon atoms and from 1 to 4 heteroatoms, for example, nitrogen, sulfur or oxygen within the ring.

The term “methylthio” refers to a moiety —S—CH₃.

The term “dihydropyridine” refers to compound A shown below, as well as to

the moieties derived from compound A:

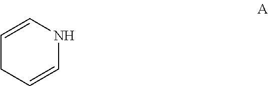

The term “benzimidazole” refers to compound B shown below, as well as to the moieties derived from compound B:

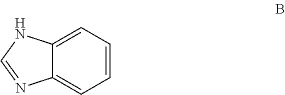

The term “phenothiazine” refers to compound C shown below, as well as to the moieties derived from compound C:

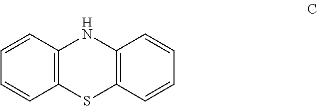

The terms “furyl,” “tetrahydrofuryl,” and “pyridyl” refer to radicals formed by removing one hydrogen from the molecules of furan, tetrahydrofuran, and pyridine, respectively.

The terms “alkyl amine” and “cyclic amine” refer to alkanes or cycloalkanes, respectively, having one hydrogen substituted by a primary, secondary or tertiary amino group, as well as to the moieties and radicals derived from such amines.

The term “alkyl amide” refers to alkanes, having one hydrogen substituted by a primary, secondary or tertiary amino group.

Each of above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)=NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

The term “alkoxy” refers to the moiety —O-alkyl, wherein alkyl is as defined above. Examples of alkoxy structures that are within the purview of the definition include, but are not limited to, (C₁-C₆)alkoxy radicals, such as methoxy, ethoxy, propoxy, isopropoxy, butoxy, iso-butoxy, sec-butoxy, tert-butoxy, pentoxy, 3-pentoxy, or hexyloxy.

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)q-U—, wherein T and U are independently —NR—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —NR—, —S(O)—, —S(O)₂—, —S(O)₂NR′—or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S(O)—, —S(O)₂—, or —S(O)₂NR′—. The substituents R, R′, R″ and R′″ are independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇cycloalkyl, and each substituted or unsubstituted heterocycloalkyl is a substituted or unsubstituted 5 to 7 membered heterocycloalkyl.

The compounds of the disclosure may exist as salts. Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When the disclosed compounds contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the disclosure contain both basic and acidic functionalities that allow the compounds to be converted into either base or acid addition salts.

The term “pharmaceutically acceptable salt” refers to salts that may be used where the compounds used in the methods of the disclosure are sufficiently basic or acidic to form stable nontoxic acid or base salts. Examples of pharmaceutically acceptable salts include organic acid addition salts formed with acids which form a physiological acceptable anion, for example, oxalate, tosylate, methanesulfonate, acetate, citrate, malonate, tartarate, succinate, benzoate, ascorbate, ketoglutarate, and glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by and are intended to be within the scope of the disclosure.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the disclosure. The compounds of the disclosure do not include those which are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another. It will be apparent to one skilled in the art that certain compounds of the disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the disclosure.

The compounds of the disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are encompassed within the scope of the disclosure.

The term “pharmaceutically acceptable salts” is meant to include salts of active compounds which are prepared with relatively nontoxic acids or bases, depending on the particular substituent moieties found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium; potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the disclosure contain both basic and acidic functionalities that allow the compounds to be converted into either base or acid addition salts.

In addition to salt forms, the disclosure provides compounds, which are in a prodrug form. Prodrugs of the compounds described herein are those compounds that readily undergo chemical changes under physiological conditions to provide the compounds of the disclosure. Additionally, prodrugs can be converted to the compounds of the disclosure by chemical or biochemical methods in an ex vivo environment. For example, prodrugs can be slowly converted to the compounds of the disclosure when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The disclosure also provides articles of manufacture comprising packaging material and a pharmaceutical composition contained within said packaging material, wherein said packaging material comprises a label which indicates that said pharmaceutical composition can be used for treatment of disorders and wherein said pharmaceutical composition comprises a compound according to the disclosure.

The disclosure also provides pharmaceutical compositions comprising at least one compound in an amount effective for treating a disorder, and a pharmaceutically acceptable vehicle or diluent. The compositions of the disclosure may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the disclosure may be formulated into therapeutic compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure include amine salts formed by the protonation of an amino group with inorganic acids such as hydrochloric acid, hydrobromic acid, hydroiodic acid, sulfuric acid, phosphoric acid, and the like. Salts of the disclosure may also include amine salts formed by the protonation of an amino group with suitable organic acids, such as p-toluenesulfonic acid, acetic acid, and the like. Additional excipients which are contemplated for use in the practice of the disclosure are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs, hydrates, and solvates of the compounds are included in the disclosure.

The disclosed pharmaceutical compositions may be administered by any suitable means, for example, orally, such as in the form of tablets, capsules, granules or powders; sublingually; buccally; parenterally, such as by subcutaneous, intravenous, intramuscular, intrathecal, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the disclosure. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The term “therapeutically effective amount” means the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the disclosure or pharmaceutical composition to the subject in need of treatment.

The pharmaceutical compositions for the administration of the compounds of this embodiment either alone or in combination with other agents, e.g., chemotherapeutic, may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Also useful as a solubilizer is polyethylene glycol, for example. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a parenterally-acceptable diluent or solvent or cosolvent or complexing agent or dispersing agent or excipient or combination thereof, for example 1,3-butane diol, polyethylene glycols, polypropylene glycols, ethanol or other alcohols, povidones, Tweens, sodium dodecyle sulfate, sodium deoxycholate, dimethylacetamide, polysorbates, poloxamers, cyclodextrins, e.g., sulfobutyl ether f3-cyclodextrin, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. For injection, the pharmaceutical compositions of the disclosure may be formulated in aqueous solutions, for example, in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

The compounds of the disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. For topical use, creams, ointments, jellies, solutions or suspensions, etc., containing the compounds of the disclosure are employed. For purposes of this application, topical application shall include mouthwashes and gargles.

In the methods described herein, an appropriate dosage level will generally be about 0.01 to 500 mg per kg patient body weight per day which can be administered in single or multiple doses. The dosage level can be about 0.01 to about 250 mg/kg per day, such as 0.01 to about 100 mg/kg per day, for example, 0.01 to about 10 mg/kg per day, such as 0.04 to about 5 mg/kg per day, or about 0.5 to about 100 mg/kg per day. A suitable dosage level may be also about 0.05 to 100 mg/kg per day, or about 0.1 to 50 mg/kg per day or 1.0 mg/kg per day. Within this range the dosage may be 0.05 to 0.5, 0.5 to 5 or 5 to 50 mg/kg per day for example. The Examples section shows that one of the exemplary compounds was dosed at 0.1 mg/kg/day while another was effective at about 1.0 mg/kg/day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or once or twice per day. There may be a period of no administration followed by another regimen of administration. Administration of the compounds may be closely associated with the schedule of a second agent of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one embodiment the disclosure provides a compound of Formula I:

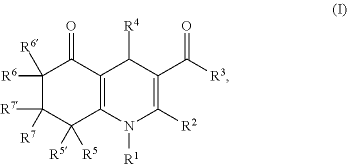

or a pharmaceutically acceptable salt or solvate thereof, wherein:

R¹ is independently hydrogen, (C₁-C₆)alkyl or a moiety forming a salt;

R² is independently hydrogen, (C₁-C₆)alkyl, CF₃ or C₂F₅;

R³ is independently OR⁸ or NR⁸R^{8′};

R⁴ is independently substituted or unsubstituted phenyl, substituted or unsubstituted pyridine, wherein phenyl or pyridine is optionally independently substituted with 1 to 3 R⁹ substituents;

R⁵, R^{5′}, R⁶, R^{6′}, R⁷, and R^{7′} are each independently hydrogen or (C₁-C₆)alkyl;

R⁸ and R^{8′} are each independently hydrogen, (C₁-C₆)alkyl, (C₃-C₈)cycloalkyl, substituted or unsubstituted heterocyclyl, aryl, (C₁-C₆)alkylaryl, or (C₁-C₆)alkylNR¹⁰R^{10′};

each R⁹ is independently hydrogen, halogen, (C₁-C₆)alkyl, (C₁-C₆)alkoxy, cyclo(C₁-C₆)alkyl, substituted or unsubstituted phenyl, substituted or unsubstituted pyridine, substituted or unsubstituted indolyl; substituted or unsubstituted pyrrolidinyl, or substituted or unsubstituted piperidinyl, wherein phenyl, pyridine, indolyl, pyrrolidinyl and piperidinyl are each optionally independently substituted with hydrogen, halogen, or (C₁-C₆)alkyl; and

R¹⁰ and R^{10′} are each independently hydrogen, (C₁-C₆)alkyl, aryl, or (C₁-C₆)alkylaryl.

In another aspect the disclosure provides a compound of Formula I, wherein R¹ is hydrogen; R² is hydrogen, CH₃ or CH₂CH₃; R³ is OR⁸; R⁴ is substituted or unsubstituted phenyl: R⁸ is hydrogen, C₁-C₆-alkyl optionally substituted by

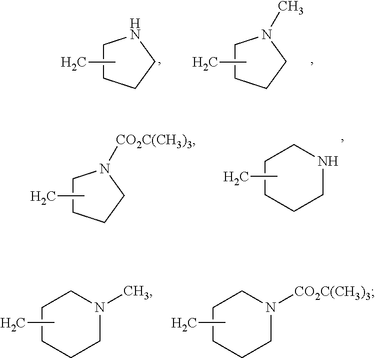

and each R⁹ is independently hydrogen, F, Cl, Br, or I.

In another aspect the disclosure provides a compound of Formula I, wherein R⁸ is CH₃, CH₂CH₃

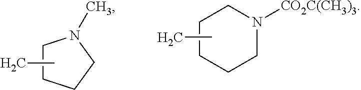

In another aspect the disclosure provides a compound of Formula IC:

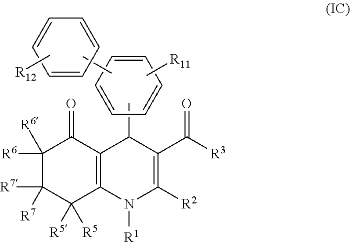

or a pharmaceutically acceptable salt or solvate thereof, wherein X is CH or N; and R¹¹ and R¹² are each independently hydrogen, halogen, or (C₁-C₆)alkyl.

In another aspect the disclosure provides a compound of Formula IC, wherein X is CH; R¹ is hydrogen; R² is hydrogen, CH₃ or CH₂CH₃; R³ is OR⁸; R⁸ is

CH₃, CH₂CH₃,

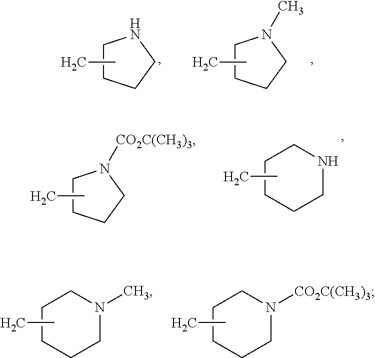

and R¹¹ and R¹² are each independently hydrogen, F, Cl, Br, I, CH₃ or CH₂CH₃.

In another aspect the disclosure provides a compound of Formula I, wherein R⁸ is CH₃, CH₂CH₃

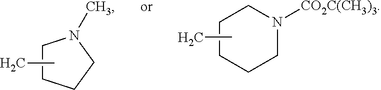

In another aspect the disclosure provides a compound of Formula ID:

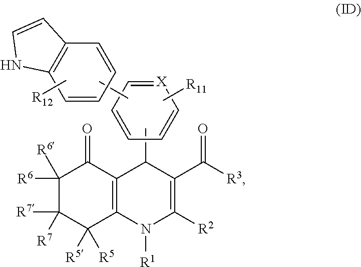

or a pharmaceutically acceptable salt thereof, wherein X is CH or N; and R¹¹ and R¹² are each independently hydrogen, halogen, or (C₁-C₆)alkyl.

In another aspect the disclosure provides a compound of Formula ID, wherein R¹ is hydrogen; R² is hydrogen, CH₃ or CH₂CH₃; R³ is OR⁸; R⁸ is CH₃, CH₂CH₃,

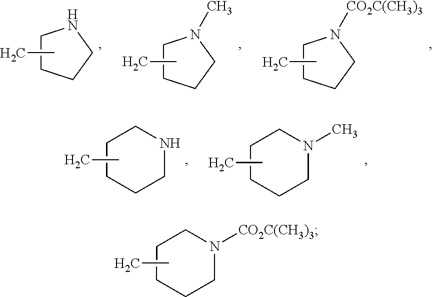

and R¹¹ and R¹² are each independently hydrogen, F, Cl, Br, I, CH₃ or CH₂CH₃.

Some specific dihydropyridine-based compounds within structure I include, but are not limited to, compounds 1-20:

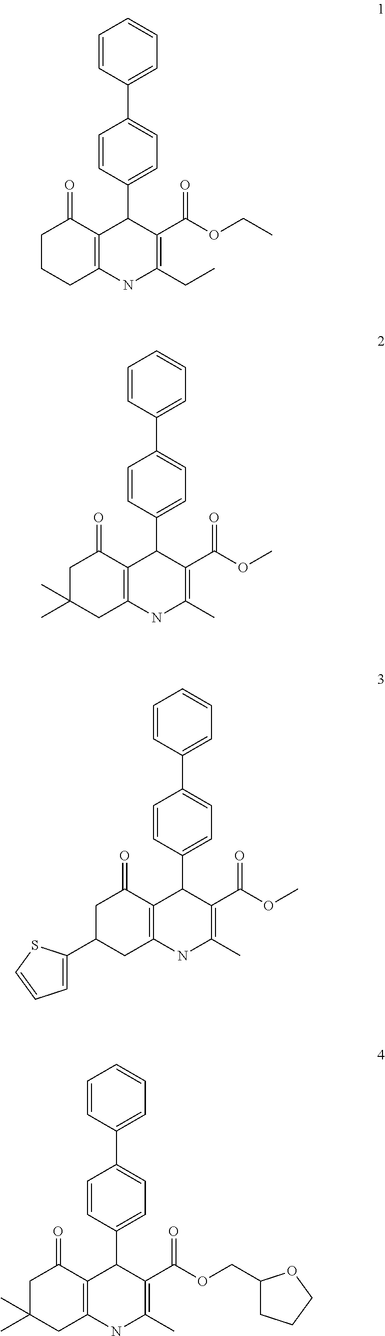

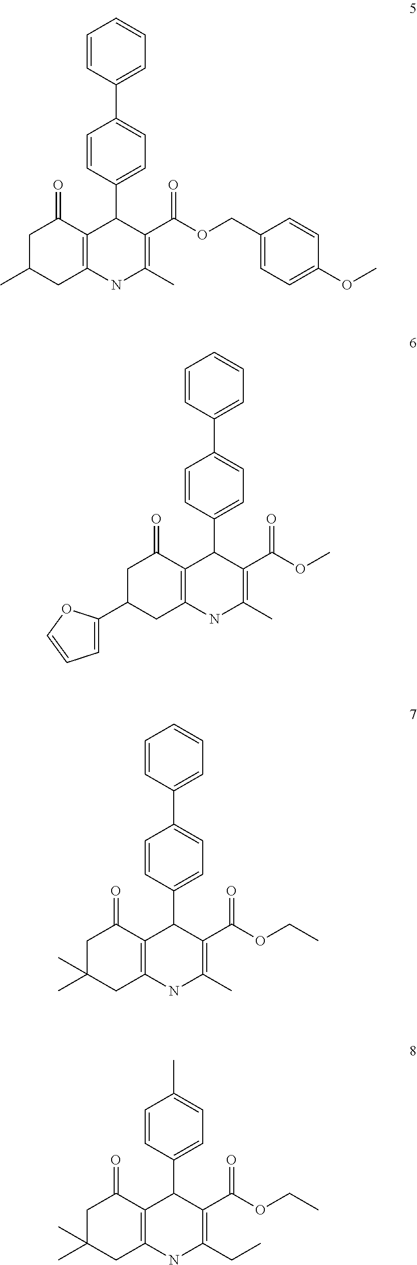

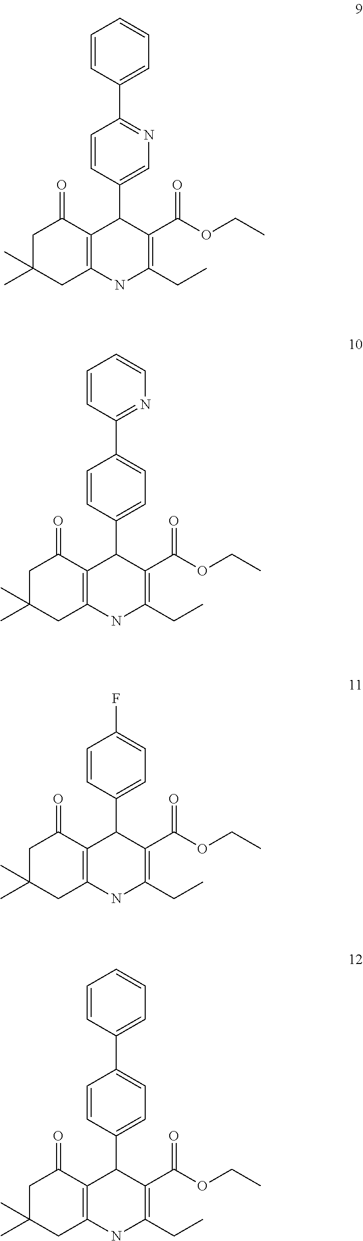

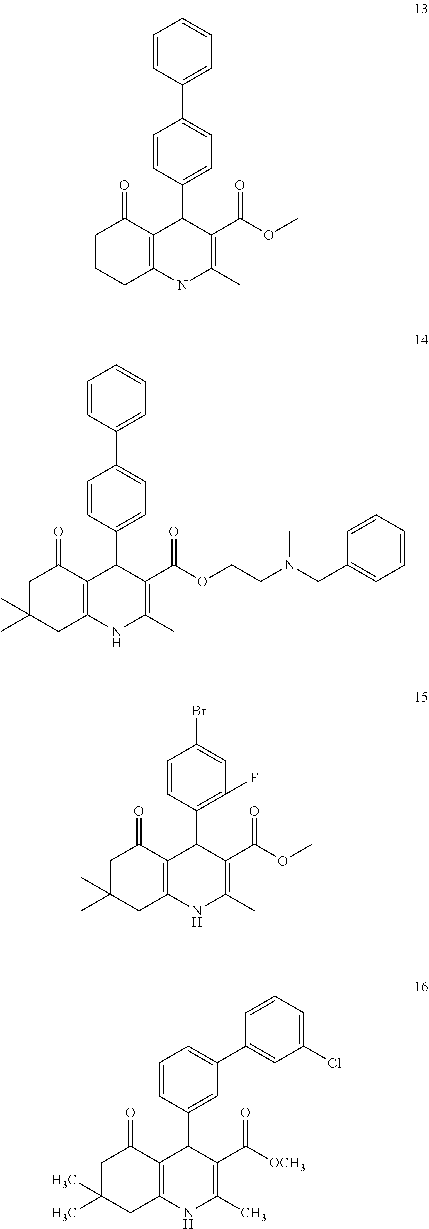

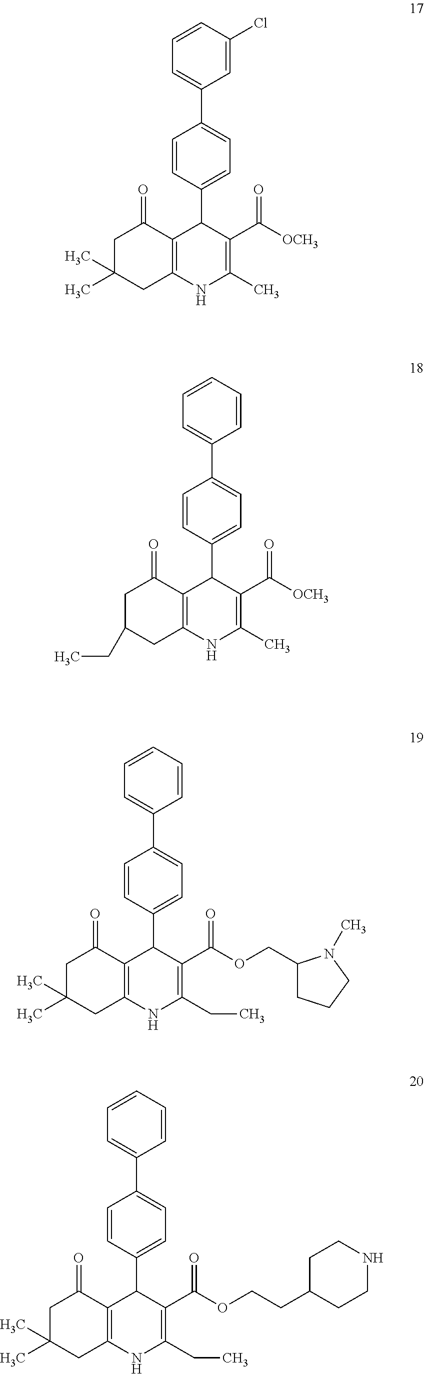

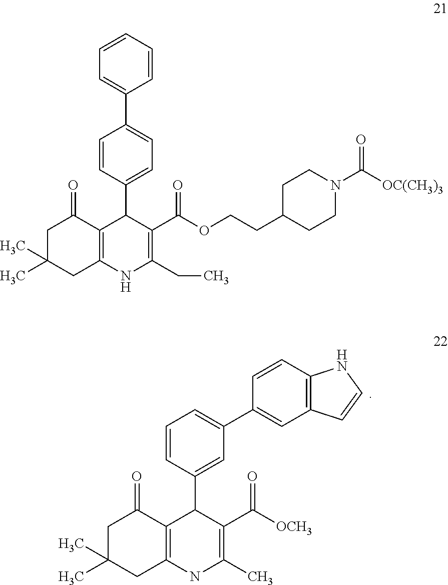

In another aspect the disclosure provides compounds of Formula I, wherein the pharmaceutically acceptable salt is the salt of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), or undecylenic acid.

In another aspect the disclosure provides methods for producing differentiated cells from stem cells by contacting the stem cells with a compound of Formula I.

In another aspect the disclosure provides methods for producing differentiated cells from stem cells by contacting the stem cells with a compound of Formula I, wherein contacting the stem cells with a compound of Formula I is from about 24 hours to about 192 hours.

In another aspect the disclosure provides methods for producing differentiated cells from stem cells by contacting the stem cells with a compound of Formula I, wherein contacting the stem cells with a compound of Formula I from about 48 hours to about 144 hours.

In another aspect the disclosure provides methods for producing differentiated cells from stem cells by contacting the stem cells with a compound of Formula I, wherein the differentiated cells are cardiomyocytes, hepatocytes or islet cells.

In another aspect the disclosure provides methods for producing differentiated cells from stem cells by contacting the stem cells with a compound of Formula I, further comprising contacting the cells with Activin A.

In another aspect the disclosure provides methods for producing differentiated cells from stem cells by contacting the stem cells with a compound of Formula I, wherein the cells differentiate to mesoderm.

In another aspect the disclosure provides methods for producing differentiated cells from stem cells by contacting the stem cells with a compound of Formula I, further comprising contacting the cells with a Wnt protein.

In another aspect the disclosure provides methods for producing differentiated cells from stem cells by contacting the stem cells with a compound of Formula I, further comprising contacting the cells with a Wnt protein, wherein the Wnt protein is Wnt3a. In other aspects the Wnt protein is Wnt5a or Wnt7.

In another aspect the disclosure provides methods for producing differentiated cells from stem cells by contacting the stem cells with a compound of Formula I, wherein the stem cells are embryonic stem cells, induced pluripotent stem cells or adult stem cells.

In another aspect the disclosure provides benzimidazole-based compounds of structure II in the form of a free base or a pharmaceutically acceptable salt, hydrate, solvate or N-oxide thereof:

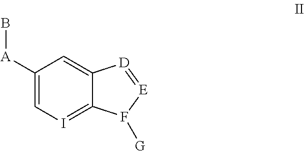

wherein A is independently NH or O; B is independently an aryl or heteroaryl moiety or a saturated 4, 5, 6 or 7 member ring optionally containing a heteroatom such as N or O, that is directly attached to A; A is a bond, a methylene, an ester, an amide, NHCH₂; D is independently N or CH, E is independently N, CH, C—R₁, C═O, C—NH₂, or C—N(R₁)₂; F is independently N or C, G is independently phenyl, aryl or cyclohexyl optionally substituted by 1 to 5 R₂; I is independently N or CH; R₁ is independently hydrogen or (C₁-C₆)alkyl; R₂ is independently hydrogen, (C₁-C₆)alkyl, (C₁-C₆)alkoxy, (C₁-C₆)thioalkoxy, hydroxy, halogen, CF₃, or C₂F₅.

In another aspect the disclosure provides methods for stem cell differentiation, comprising contacting the embryonic stem cells with a compound of structure I in the form of a free base or a pharmaceutically acceptable salt, hydrate, solvate or N-oxide thereof, wherein A, B, D, E, F, G, and I are as described above.

In another aspect the disclosure provides benzimidazole-based compounds of structure II, including 1-phenyl-5-(1-phthalazinoamino)-benzimidazole:

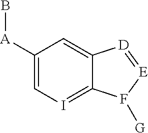

wherein A is a bond, N, O, NHCH₂, COO, CONH, or NHCO linker; B is an aromatic ring, heterocycle, or optionally substituted alkyl; D is N or CH; E is CH, CR₁, C═O, C═S, or N; R₁ is an optionally substituted alkyl or an optionally substituted amine; G is an aromatic ring or a heterocycle; and I is C or N.

The compound of structure II includes the structure: 1-phenyl-5-(1-arylamino)-benzimidazole in the form of a free base or a pharmaceutically acceptable salt, hydrate, solvate or N-oxide thereof:

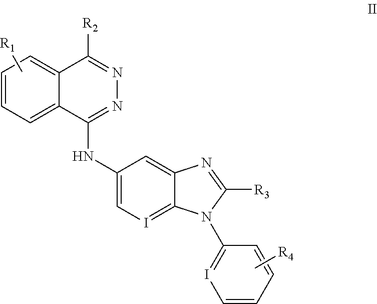

wherein R₁ is H, CH₃; R₂ is (C₁-C₆)alkyl, halogen, methoxy, benzyl; R₃ is H, (C₁-C₆)alkyl, amine optionally substituted by (C₁-C₆)alkyl, R₄ is H, CH₃, OCH₃, SCH₃, CF₃, (C₁-C₆)alkyl, halogen, alkoxy; and I is CH, N.

In another aspect the disclosure provides benzimidazole-based compounds of structures II, HA, IIB, IIC, IID, IIE, IIF, IIG, IIH, IIJ, IIK, IIL, and IIM in the form of free base or a pharmaceutically acceptable salt, hydrate, solvate or N-oxide thereof:

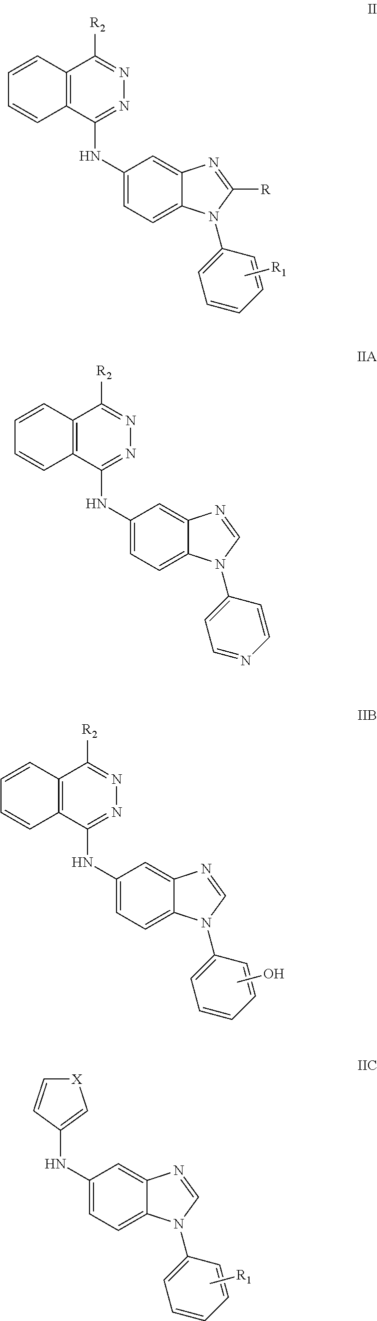

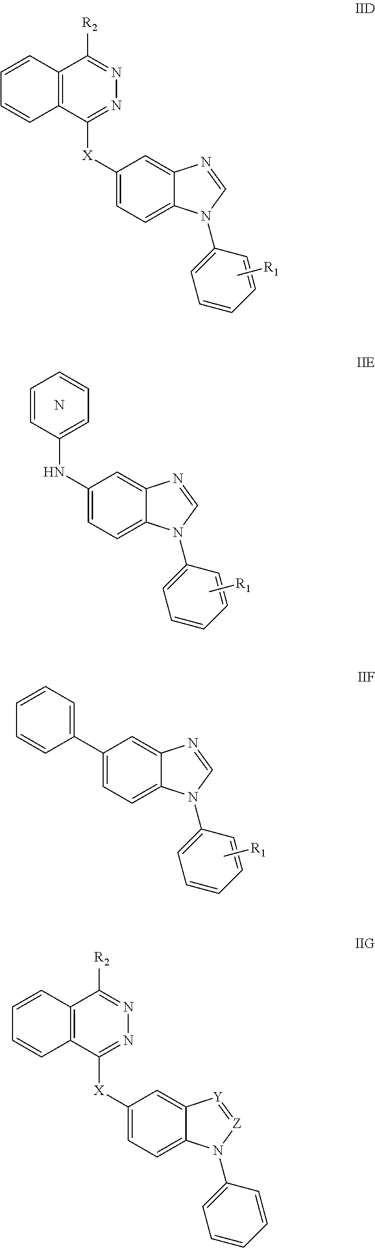

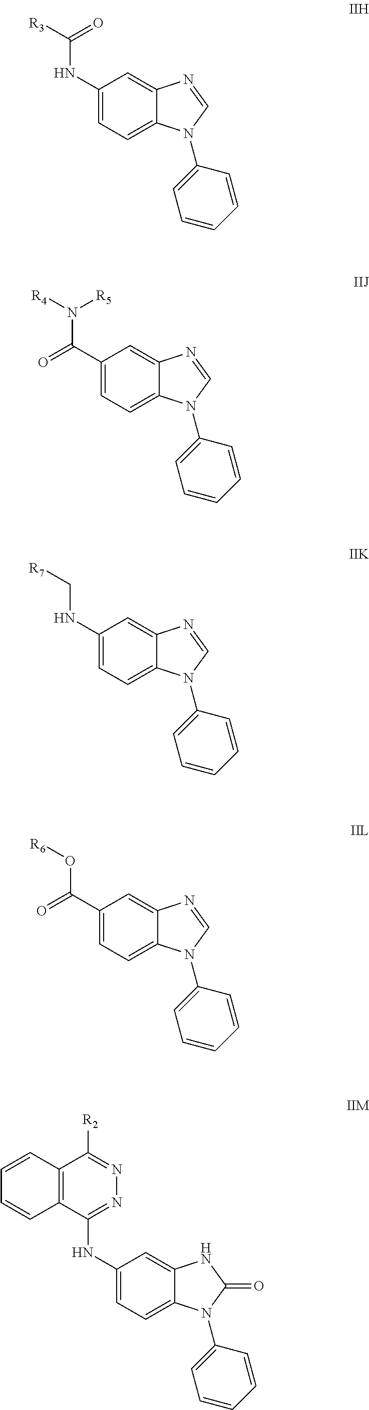

wherein R is independently hydrogen, methyl, or amino; R₁ is independently hydrogen, hydroxyl, pyridyl, methyl, trifluoromethyl, methoxy or methylthio; R₂ is independently hydrogen, phenyl, benzyl, methoxy, methyl or halogen; R₃, R₄, R₅, R₆, R₇ are C₁-C₆ alkyl, optionally substituted phenyls or heteroaryls, each of Y and Z is independently CH or N; and X is independently CH₂, NH, O, S, S═O, SO₂, CH(OH), or C═O,

Some specific benzimidazole-based disclosure compounds within structures II, IIA, IIB, IIC, IID, IIE, IIF, IIG, IIH, IIJ, IIK, IIL, and IIM include, but are not limited to compounds 20-42:

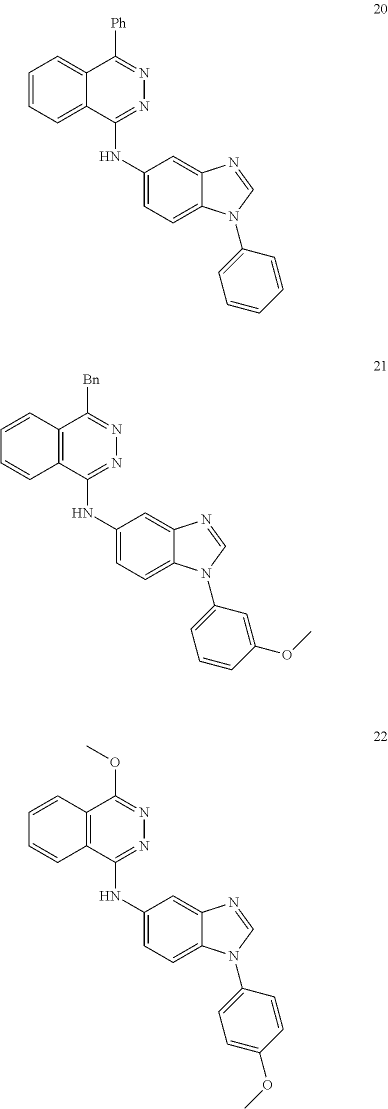

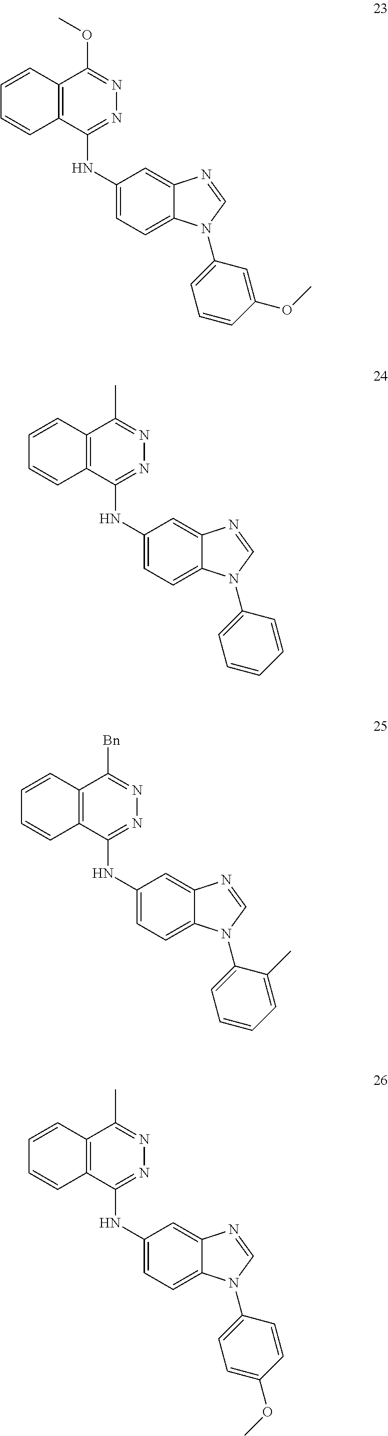

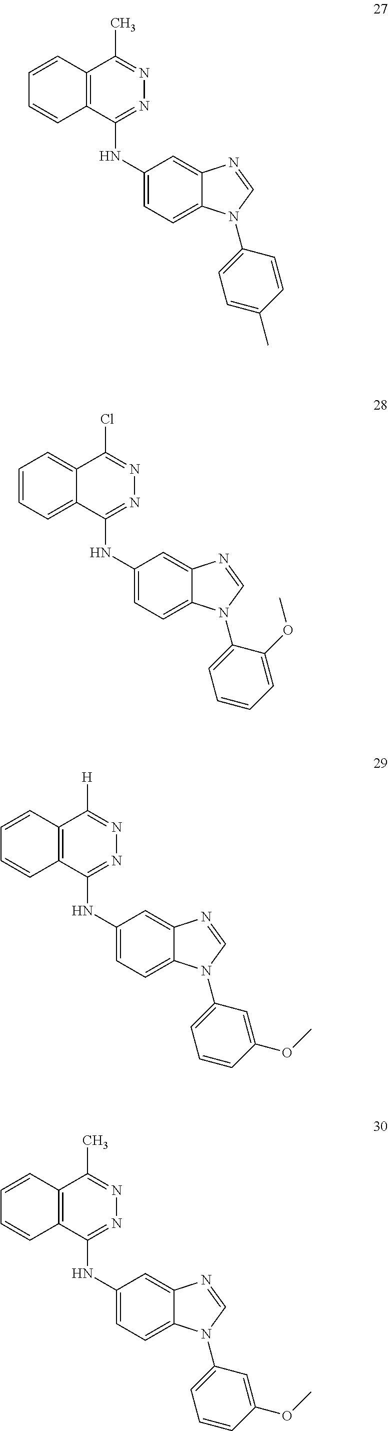

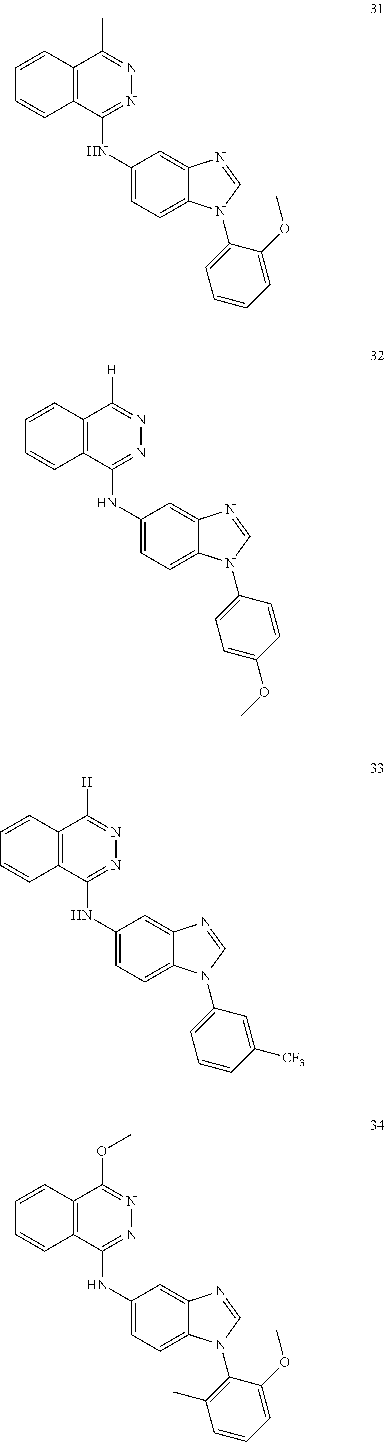

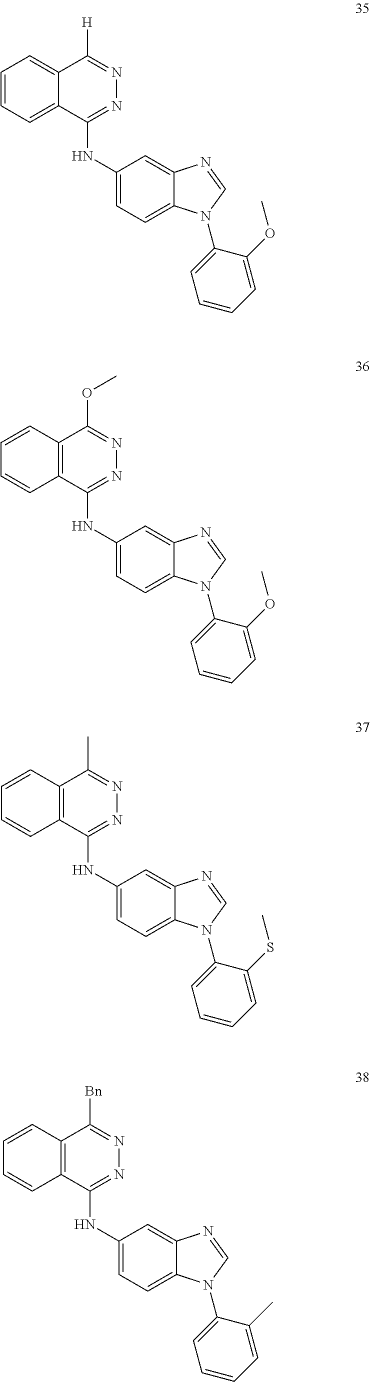

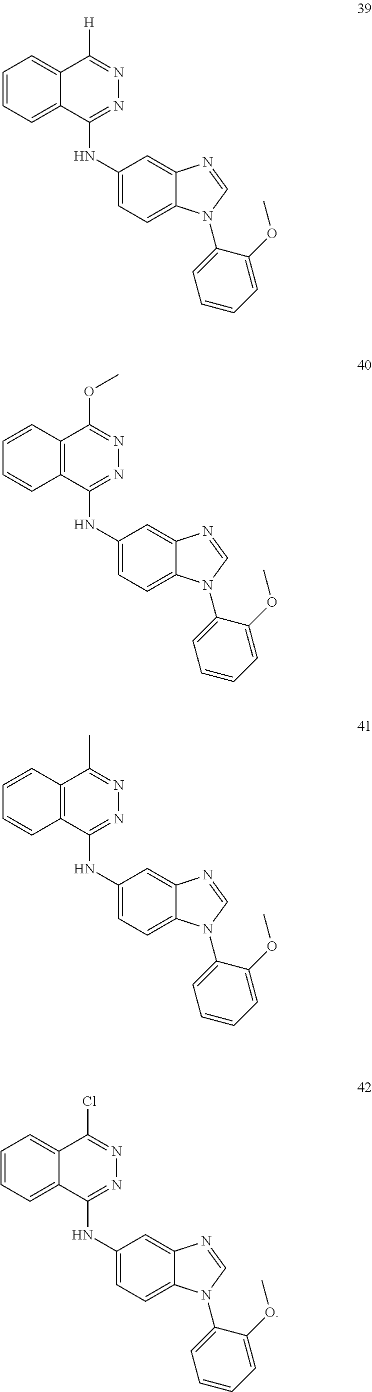

In another aspect the disclosure provides phenothiazine-based compounds of structure IX in the form of free base or a pharmaceutically acceptable salt, hydrate, solvate or N-oxide thereof:

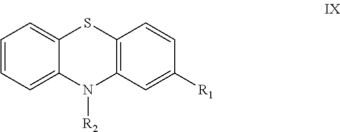

wherein R₁ is independently CF₃ and chloro; and R₂ is independently an alkyl amine, a cyclic amine, an aliphatic cyclic amine, or an alkyl amide.

Some specific phenothiazine-based compounds within structure IX include, but are not limited to, compounds 43-48:

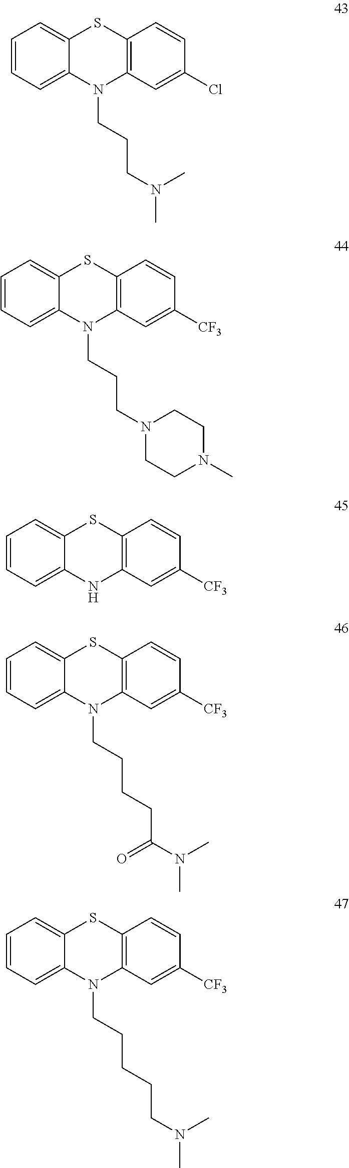

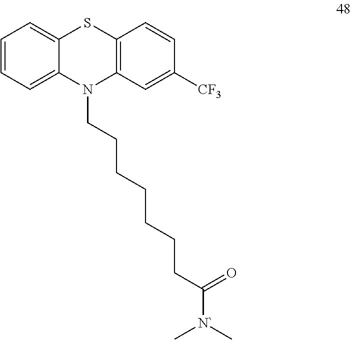

The compounds of structure IV include 1-alkylamino-2′-substituted diphenylamine IVa:

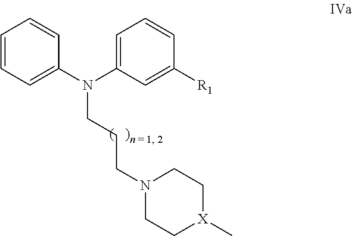

wherein R₁ is H, CH₃, halogen; X is CH, N, or O.

In another aspect the disclosure provides methods for producing differentiated cells from stem cells. The methods comprise contacting stem cells with the disclosed compounds that stimulate the production of differentiated cells thereby. The disclosed compounds may be used to carry out such methods include all the compounds within the above-described genera and sub-general I, II, HA, IIB, IIC, IID, IIE, IIF, IIG, IIH, IIK, ILL, IIM, IVa and IX, including particular species 1-48, also described above.

In another aspect the disclosure provides tamoxifen-based compounds of structure VI, VII or VIII in the form of free base or a pharmaceutically acceptable salt, hydrate, solvate or N-oxide thereof:

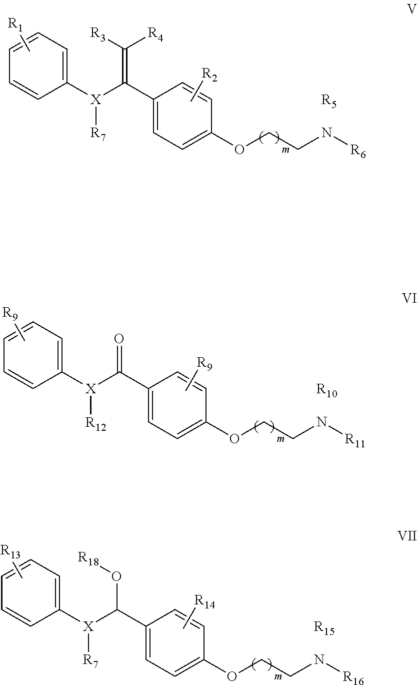

wherein for compound VI, X is independently a bond, CH₂, or CHR₇; R₁ is independently methyl, ethyl, (C₁-C₆)alkyl, halogen, phenyl, methoxy, phenoxy, nitro, trifluoromethyl, or alkylamino; R₂ is independently methyl, ethyl, phenyl, (C₁-C₆)alkyl, trifluoromethyl, or halogen; R₃ is independently methyl, ethyl, (C₁-C₆)alkyl, phenyl, or aryl; R₄ is independently methyl, ethyl, (C₁-C₆)alkyl, phenyl, or aryl; R₅ is independently methyl, ethyl, (C₁-C₆)alkyl, phenyl, or aryl; R₆ is independently methyl, ethyl, (C₁-C₆)alkyl, phenyl, or aryl, wherein R₅ and R₆ may be joined via a ring, R₇ is independently methyl, ethyl, propyl, (C₁-C₆)alkyl, phenyl, or benzyl; and m is independently 0-4 methylene units; and wherein for compound VII, X is independently a bond, CH₂, or CHR₁₂; R₈ is independently methyl, ethyl, (C₁-C₆)alkyl, halogen, phenyl, methoxy, phenoxy, nitro, trifluoromethyl, or alkylamino, R₉ is independently methyl, ethyl, phenyl, (C₁-C₆)alkyl, trifluoromethyl, or halogen; R₁₀ is independently methyl, ethyl, (C₁-C₆)alkyl, phenyl, or aryl, R₁₁ is independently methyl, ethyl, (C₁-C₆)alkyl, phenyl, or aryl, wherein R₁₀ and R₁₁ can be joined via a ring; R₁₂ is independently methyl, ethyl, propyl, (C₁-C₆)alkyl, phenyl, or benzyl; and m is independently 0-4 methylene units; and wherein for compound VIII, X is independently a bond, CH₂, or CHR₁₇; R₁₃ is independently methyl, ethyl, (C₁-C₆)alkyl, halogen, phenyl, methoxy, phenoxy, nitro, trifluoromethyl, or alkylamino; R₁₄ is independently methyl, ethyl, phenyl, (C₁-C₆)alkyl, trifluoromethyl, or halogen; R₁₅ is independently methyl, ethyl, (C₁-C₆)-alkyl, phenyl, or aryl; R₁₆ is independently methyl, ethyl, (C₁-C₆)alkyl, phenyl, or aryl, wherein R₁₅ and R₁₆ may be joined via a ring; R₁₇ is independently methyl, ethyl, propyl, (C₁-C₆)alkyl, phenyl, or benzyl; R₁₈ is independently methyl, ethyl, propyl, (C₁-C₆)alkyl, phenyl, or benzyl; and m is independently 0-4 methylene units.

In another aspect the disclosure provides methods for stem cell differentiation, comprising contacting the embryonic stem cells with a compound of structure VI, VII or VIII, in the form of free base or a pharmaceutically acceptable salt, hydrate, solvate or N-oxide thereof.

Those skilled in the art may determine the optimal time of contacting the stem cells and with the disclosed compounds described below required achieving the optimal results. As a guideline, the period of contact may be between about 24 hours and about 192 hours, for example, between about 48 hours and about 144 hours. Differentiated cells produced by the disclosed methods may include are cardiomyocytes, liver cells, lung cells, pancreatic cells, and others.

The stem cells suitable for use in the disclosed methods may be derived from a patient's own tissue. This would enhance compatibility of differentiated tissue grafts derived from the stem cells with the patient. In this context it should be noted that embryonic stem cells can include adult stem cells derived from a person's own tissue iPSCs, embryonic stem cells, and the like. Human stem cells may be genetically modified prior to use through introduction of genes that may control their state of differentiation prior to, during or after their exposure to the embryonic cell or extracellular medium from an embryonic cell. They may be genetically modified through introduction of vectors expressing a selectable marker under the control of a stem cell specific promoter, such as Oct-4. The stem cells may be genetically modified at any stage with a marker so that the marker is carried through to any stage of cultivation. The marker may be used to purify the differentiated or undifferentiated stem cell populations at any stage of cultivation.

The disclosure also provides differentiated cells produced according to the disclosed methods that may be used for transplantation, cell therapy or gene therapy. The disclosure further provides a differentiated cell produced according to the disclosed methods that may be used for therapeutic purposes, such as in methods of restoring cardiac function in a subject suffering from a heart disease or condition.

In another aspect the disclosure provides methods of treating or preventing a cardiac disease or condition, the method including introducing an isolated differentiated cardiomyocyte cell of the disclosure and/or a cell capable of differentiating into a cardiomyocyte cell when treated in accordance with the disclosed methods into cardiac tissue of a subject. The isolated cardiomyocyte cell may be transplanted into damaged cardiac tissue of a subject. The method may result in the restoration of cardiac function in a subject.

In another aspect the disclosure provides methods of repairing cardiac tissue, the method including introducing an isolated cardiomyocyte cell of the disclosure and/or a cell capable of differentiating into a cardiomyocyte cell when treated in accordance with the method of the disclosure into damaged cardiac tissue of a subject.

The subject may be suffering from a cardiac disease or condition. In the method of the disclosure, the isolated cardiomyocyte cell may be transplanted into damaged cardiac tissue of a subject. The method may result in the restoration of cardiac function in a subject. The disclosure also provides a myocardial model for testing the ability of stem cells that have differentiated into cardiomyocytes to restore cardiac function. The disclosure further provides a cell composition including a differentiated cell of the disclosure, and a carrier. The term “inducing differentiation” as used herein is taken to mean causing a stem cell to develop into a specific differentiated cell type as a result of a direct or intentional influence on the stem cell. Influencing factors in addition to the compounds described herein can include cellular parameters such as ion influx, a pH change and/or extracellular factors, such as secreted proteins, such as but not limited to growth factors and cytokines that regulate and trigger differentiation. It may include culturing the cell to confluence and may be influenced by cell density.

The SC and the cell providing the differentiating factor(s) may be co-cultured in vitro. This typically involves introducing the stem cell to an embryonic cell monolayer produced by proliferation of the embryonic cell in culture.

The cellular and molecular events regulating the induction and tissue-specific differentiation of endoderm are important to understanding the development and function of many organ systems. Stem cell-derived endoderm is important for the development of cellular therapies for the treatment of disease such as diabetes, liver cirrhosis, or pulmonary emphysema (e.g., via development of islet cells, hepatocytes or lung cells, respectively). Accordingly, compounds described in the disclosure find particular use in inducing differentiation of cells in the endoderm lineage, including pancreas, liver, lung and the like.

In one aspect, the compounds of the disclosure are used to screen for targets of their action. For example, competitive analyses can be performed using compounds with known targets. Such targets include, for example, but not limited to MEF2C; Beta-catenin; TCF/LEF; Smad2, Smad3; Smad4 (binding partners of the above proteins are also potential targets since they would modulate activity); p38, and components of the signaling that activate MEF2C; components of the Wnt pathway, such as Frizzled proteins, CaMK, Axin, Dishevelled, APC, GSK3, FRAP; Calmodulin (in particular for phenothiazine analogues); Potassium channel targets (in particular for dihydropyridine analogues); and Calcium channel targets (in particular for dihydropyridine analogues).

### EXAMPLES

The embodiments of the disclosure may be further illustrated by the following non-limiting examples.

### Example 1

### Biological Assays

The primary screen is conducted with CGR8 mESCs stably transfected with eGFP under control of the alpha myosin heavy chain (aMHC) promoter (Takahashi, et al., Circulation, 107(14):1912, 2003). The bioassay is run essentially as described (Bushway et al., Methods Enzymol, 414:300, 2006). Briefly, cells were seeded onto Greiner 384 well microclear bottom microtiter plates in ½ well volume at a density of ˜229 cells/mm2. Compounds are administered on day 2 with ½ well volume at 2× concentration and mixed thoroughly with replacement on day 4 by aspiration and replacement of 1× concentrated compound in ½ well volume; otherwise, fresh media is replaced at well volume every second day until the assay is complete. The primary assay is executed on the Beckman FX with robotic arm and integrated cytomats using SAMI scheduler.

The optimal time to stop the differentiation is empirically determined to be at day 9 of differentiation, when cardiomyocytes appear in positive control cultures that have higher density cells or culture the cells in embryoid body (not monolayer) culture. Plates were fixed for 5 minutes in 4% paraformaldehyde in 1×PBS, rinsed 3 times in 1×PBS (includes a DAPI stain). 50% glycerol is then added to each well and plates stored until imaging. A total of 30,000 data points were obtained. That is ˜14,000 unique compounds were screened at 1 μg/mL and 5 μg/mL, corresponding to approximately 2 μM and 13 μM (assuming approximate MW 300-500 g/mole). The primary screen imaging is done with Q3DM Eidaq 100 mounted with a 4× objective capturing 4 images/well at 8×8 binning. Plates were loaded manually. Image quantification is done using a simple image subtraction routine that subtracted the red channel images from the green (eGFP) channel images to remove background signal from the eGFP images. This algorithm yielded an integrated value for each well.

Follow-up confirmations and testing of hits for SAR were performed on the Hamilton STAR fluid handler with integrated Kendro Cytomat plate hotel and Kendro Cytomat plate incubators using the Hamilton STAR liquid handler robot. By the time of these later experiments, our imaging infrastructure and algorithms had changed. Imaging is done on the INCell 1000 (GE/Amersham) using a 10× objective capturing 9 images/well at 4×4 binning during image capture. Microtiter plate loading is automated using the CRS/Thermo Catalyst Express robotic arm and Polara scheduler. Image quantification is performed on captured TIFF images using the Developer Toolbox (GE/Amersham) with custom algorithms.

In brief, each image is dynamically thresholded by acquiring a global pixel average and multiplying this value by a scalar to produce an image mask approximating the specific signal. The mask is used to collect integrated intensities in blue (DAPI), green (eGFP), and red (non-specific) channels. Typical data treatment subtracts integrated intensities of the red channel from the specific signal in the green channel. In dose response curves for SAR studies, each compound is tested in a 5-step, 2-fold titration observing a minimum of 4 replicates wells/titer, or 36 separate images.

### Example 2

### General Synthetic Procedures for Obtaining Compounds of Formula I

The dihydropyridine-based compounds of general structure IA and IB:

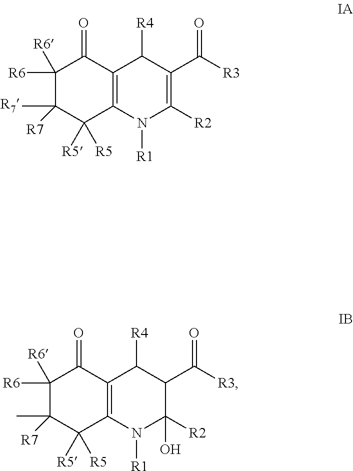

may be synthesized according to Scheme 1:

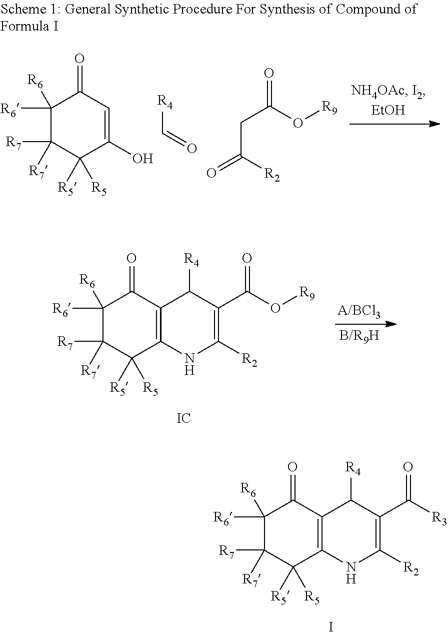

Compound IC: To a 10 mL flask, 1 eq. of the appropriate 1,3-dione, 1 eq. of the appropriate aldehyde, 1 eq. of the appropriate ketoacetate, 1 eq. of ammonium acetate, and 0.3 eq. of iodine were added to a minimum amount of ethanol to produce a slurry and stirred at rt. After stirring overnight the reaction mixture was diluted with ethyl acetate and washed with an aqueous solution of sodium thiosulfate. The organic layers were combined and dried over sodium sulfate, concentrated in vacuo to give a crude solid. The crude material was purified by silica gel chromatography.

When R₃ is different from methyl or ethyl, the methyl ester, intermediate I is hydrolyzed in the presence of boron trichloride and then reacted in the presence of the desired alcohol or amine using standard techniques to give compound I.

Compound I: Excess of boron trichloride was added to a cold solution of Compound IC in dry dichloromethane. The mixture was stirred at rt overnight. The reaction was stopped by pouring the mixture in ice water and extracted with ethyl acetate. Purification by chromatography gave the acid. The acid is then activated with thionyl chloride in presence of catalytic amounts of N,N-dimethylformamide in dichloromethane followed by reaction with the desired alcohol or amine.

According to Scheme 2, the Compound of Formula III may be prepared as follows

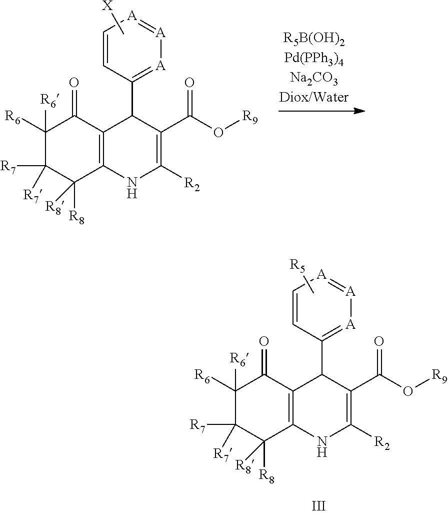

To a microwave vial was added 0.05 mmol of Compound IC, 0.06 mmol of the appropriate boronic acid, 0.006 mmol of tetrakis(triphenylphosphine)palladium(0), 1.2 mmol of sodium carbonate (2M in water), and 0.5 mL of 1:1 water/dioxane. The vial was sealed and heated at 150° C. for 10 min in a microwave. The reaction mixture was diluted water and extracted with ethyl acetate. The crude material was purified by column chromatography to afford the desired product.

Synthetic Procedures and Analytical Data for Compounds 15-22.

### Compound 22: Methyl 4-(3-(1H-indol-5-yl)phenyl)-2,7,7-trimethyl-5-oxo-1,4,5,6,7,8-hexahydroquinoline-3-carboxylate

Intermediate 1: Methyl 4-(3-bromophenyl)-2,7,7-trimethyl-5-oxo-1,4,5,6,7,8-hexahydro-quinoline-3-carboxylate: To a 10 mL flask, 7.1 mmol (1 g) of 5-dimethyl-1,3-cyclohexyldione, 7.1 mmol (1.3 g) of 3-bromobenzaldehyde, 7.1 mmol (0.82 mL) of methyl acetoacetate, 7.1 mmol (547 mg) of ammonium acetate, and 2.1 mmol (543 mg) of iodine were added to 3 mL of ethanol and stirred at rt. After stirring overnight the reaction mixture was diluted with 200 mL ethyl acetate and washed with an aqueous solution of sodium thiosulfate. The organic layers were combined and dried over sodium sulfate, concentrated in vacuo to give a crude solid. The crude material was recrystallized from ethanol to give a yellow solid (1.13 g). ¹H NMR (300 MHz DMSO-d6); δ 0.72 (3H, s), 0.86 (3H, s), 1.93-1.97 (2H, m), 2.11-2.14 (2H, m), 2.16 (3H, s), 3.39 (3H, s), 4.78 (1H, s), 6.83-6.88 (1H, m), 6.97-7.05 (2H, m), 7.14-7.16 (1 H, m), 8.14 (1H, bs).

To a microwave vial was added 0.05 mmol of intermediate 1, 0.06 mmol of the appropriate boronic acid, 0.006 mmol of tetrakis(triphenylphosphine)palladium(0), 1.2 mmol of 2 M sodium carbonate, and 0.5 mL of 1:1 water/dioxane. The vial was sealed and heated at 150° C. for 10 min. The reaction mixture was diluted water and extracted with ethyl acetate. The crude material was purified by column chromatography to give the expected product (hexanes/ethyl acetate 1/1; R_{f}=0.78). MS: 463.27 [M+Na]. ¹H NMR (300 MHz CD₃OD); δ 0.93 (3H, s), 1.07 (3H, s), 2.07-2.14 (2H, m), 2.23-2.47 (2H, m), 2.36 (3H, s), 3.61 (3H, s), 5.03 (1H, s), 6.46 (1H, s), 7.12-7.14 (1H, m), 7.19-7.24 (2H, m), 7.19-7.24 (3H, m), 7.53 (1H, s), 7.693 (1H, s). 10.46 (1H, bs).

Compound 12: Ethyl 4-(biphenyl-4-yl)-2-ethyl-7,7-dimethyl-5-oxo-1,4,5,6,7,8-hexahydro-quinoline-3-carboxylic ester

To a 10 mL flask, 15.4 mmol (2.15 g) of 5,5-dimethyl-1,3-cyclohexyldione, 15.4 mmol (2.8 g) of 4-phenylbenzaldehyde, 15.4 mmol (2 mL) of methyl propionylacetate, 15.4 mmol (1.2 g) of ammonium acetate, and 5.1 mmol (586 mg) of iodine were added to 3 mL of ethanol and stirred at rt. After stirring overnight the reaction mixture was diluted 200 mL ethyl acetate and washed with an aqueous solution of sodium thiosulfate. The organic layers were combined and dried over sodium sulfate, concentrated in vacuo to give a crude solid. The crude material was purified by liquid chromatography (silica gel, hexanes/ethyl acetate 1/1) to give a pale yellow solid (2.8 g). ¹H NMR (300 MHz, CDCl₃); δ 0.93 (3H, s), 1.08 (3H, s), 1.25 (3H, t, J=7.8 Hz), 2.18-2.41 (3H, m), 2.81 (2H, q, J=7.8 Hz), 3.63 (3H, s), 5.98 (1H, bs), 7.26-7.45 (7H, m), 7.53 (2H, bd, J=8.1 Hz).

Compound 21: 2-(1-(tert-butoxycarbonyl)piperidin-4-yl)ethyl 4-(biphenyl-4-yl)-2-ethyl-7,7-dimethyl-5-oxo-1,4,5,6,7,8-hexahydroquinoline-3-carboxylate:

4-(Biphenyl-4-yl)-2-ethyl-7,7-dimethyl-5-oxo-1,4,5,6,7,8-hexahydroquinoline-3-carboxylic acid: 10 mL of a 1M solution of boron trichloride was added to a cold solution of 300 mg (0.72 mmol) of methyl 4-(biphenyl-4-yl)-2-ethyl-7,7-dimethyl-5-oxo-1,4,5,6,7,8-hexahydro-quinoline-3-carboxylic ester in 10 mL dry dichloromethane. The mixture was stirred at rt overnight. The reaction was checked by TLC (dichloromethane/methanol 9/1), quenched by pouring the mixture in ice water and extracted with ethyl acetate. Purification by chromatography (dichloromethane with methanol 0 to 10%) gave 280 mg of an off-white solid. ¹H NMR (300 MHz DMSO-d6); δ 0.81 (3H, s), 0.98 (3H, s), 1.91-2.80 (2H, m), 4.87 (1H, s), 7.16-7.24 (1H, m), 7.33-7.54 (2H, m), 7.53-7.62 (1H, m), 9.01 (1H, bs).

Compound 21: 35 mg of 4-(biphenyl-4-yl)-2-ethyl-7,7-dimethyl-5-oxo-1,4,5,6,7,8-hexahydro quinoline-3-carboxylic acid was dissolved in 3 mL dichloromethane. 1 drop of N,N-dimethylformamide and 20 microL of oxalyl chloride was added. After a hour at rt, solvents were evaporated, the residue dissolved in 4 mL of dichloromethane with 20 μL of DIEA and 40 mg of 1-(tert-butoxycarbonyl)piperidin-4-yl)ethanol was added. The mixture was stirred at rt overnight. The reaction was checked by TLC (dichloromethane/methanol 9/1). The crude mixture was purified by PTLC with hexane/ethyl acetate 7/3 (R_{f} 0.6) to give a bright yellow solid (15 mg).

### Compound 20: 2-(Piperidin-4-yl)ethyl 4-(biphenyl-4-yl)-2-ethyl-7,7-dimethyl-5-oxo-1,4,5,6,7,8-hexahydro-quinoline-3-carboxylate

5 mg of compound 21 was dissolved in a 1M HCl solution in diethyl ether and stirred at rt for one hour. TLC (hexane/ethyl acetate 7/3) showed complete deprotection. Solvent was evaporated and the product used without any purification. MS: 471.07 [M+H].

Compound 15: Methyl 4-(4-bromo-2-fluorophenyl)-2,7,7-trimethyl-5-oxo-1,4,5,6,7,8-hexa-hydroquinoline-3-carboxylate: Following the procedure described for intermediate 1 using 4-bromo-2-fluoro benzaldehyde, 46 was obtained as a light yellow powder (21% yield). MS: 421.07 [M+H]. ¹H NMR (300 MHz DMSO-d6); δ 0.82 (3H, s), 0.99 (3H, s), 1.88-1.93 (1H, m), 2.12-2.26 (5H, m), 2.38-2.44 (1H, m), 3.47 (3H, s), 4.98 (1H, s), 7.09-7.15 (1H, m), 7.23-7.31 (2H, m), 9.15 (1H, bs).

### Compound 17: Methyl 4-(4-(3-chlorophenyl)phenyl)-2,7,7-trimethyl-5-oxo-1,4,5,6,7,8-hexahydro-quinoline-3-carboxylate

Methyl 4-(4-bromophenyl)-2,7,7-trimethyl-5-oxo-1,4,5,6,7,8-hexahydro-quinoline-3-carboxylate: Following the procedure described for intermediate 1 and using 4-bromobenzaldehyde the expected product was obtained as a yellow solid (25% yield). ¹H NMR (300 MHz DMSO-d6); δ 0.81 (3H, s), 0.99 (3H, s), 1.93-1.99 (1H, m), 2.14-2.23 (3H, m), 2.28 (3H, s), 3.50 (3H, s), 4.81 (1H, s), 7.08 (2H, d, J=8.3), 7.37 (2H, d, J=8.3), 9.15 (1H, bs). The next step, the Suzuki coupling was performed as previously described for compound 44 and using 3-chlorophenyl boronic acid. MS: 458.2 [M+Na].

Compound 18: Methyl 4-(biphenyl-4-yl)-7-ethyl-2-methyl-5-oxo-1,4,5,6,7,8-hexahydro-quinoline-3-carboxylate: Following the procedure described for intermediate 1 and using 4-phenylbenzaldehyde the expected product was obtained as a white solid. MS: 424.07 [M+Na].

### Compound 19: R-(1-methylpyrrolidin-2-yl)methanol 4-(biphenyl-4-yl)-2-ethyl-7,7-dimethyl-5-oxo-1,4,5,6,7,8-hexahydroquinoline-3-carboxylate

Intermediate 19: To a solution of 50 mg (0.49 mmol) of R-prolinol in ethanol was added 0.28 ml of formaldehyde (37% wt/w) and 0.44 ml of borane-pyridine complex. The mixture was stirred at rt overnight, solvents were evaporated and the crude R—N-methyl prolinol was used as is in the next step. 1.2 mmol (0.1 mL) of diketene, 1.5 mmol R-(1-methylpyrrolidin-2-yl)methanol 1, 2 drops of TEA were added to 2 mL of dichloromethane and stirred overnight at rt Solvents were evaporated and the product, (1-methylpyrrolidin-2-yl)methanol was used in the next step.

Compound 19: 14 mg (1 mmol) of 5,5-dimethyldimedone, 18 mg (1 mmol) of 4-phenylbenzaldehyde, 20 mg (1 mmol) of intermediate 49, 8 mg (1 mmol) of ammonium acetate, 13 mg (0.3 mmol) of iodine were stirred overnight in a sealed vial with 5 drops of ethanol. Solvent was evaporated, the residue dissolved in 4 mL of dichloromethane, washed with 1 ml of water and the organic layer was purified by PTLC (dichloromethane/methanol Rf 0.5). ¹H NMR (300 MHz CD₃OD) 0.91 (3H, s), 1.07 (3 H, s), 1.95-2.51 (8 μl, m), 2.42 (3H, s), 2.91 (s, 3H), 3.19-3.65 (m, 3H), 3.31 (3H, s), 5.01 (1H, s), 5.21-5.38 (m, 1H), 7.26-7.42 (4H, m), 7.45-7.57 (3H, m). MS: 471.07 [M+H].

Compound 16: methyl 4-(3-(3-chloro)phenyl)-2,7,7-trimethyl-5-oxo-1,4,5,6,7,8-hexahydro-quinoline-3-carboxylate: Following procedure described for compound I. MS: 458.07 [M+Na].

Compound 21: 2-(1-(tert-butoxycarbonyl)piperidin-4-yl)ethyl 4-(biphenyl-4-yl)-2-ethyl-7,7-dimethyl-5-oxo-1,4,5,6,7,8-hexahydroquinoline-3-carboxylate: 35 mg of 4-(biphenyl-4-yl)-2-ethyl-7,7-dimethyl-5-oxo-1,4,5,6,7,8-hexahydroquinoline-3-carboxylic acid was dissolved in 3 mL dichloromethane. 1 drop of N,N-dimethylformamide and 20 microL of oxalyl chloride was added. After a hour at rt, solvents were evaporated, the residue dissolved in 4 mL of dichloromethane with 20 μL of DIEA and 40 mg of 1-(tert-butoxycarbonyl)piperidin-4-yl)ethanol was added. The mixture was stirred at rt overnight. The reaction was checked by TLC (dichloromethane/methanol 9/1). The crude mixture was purified by PTLC with hexane/ethyl acetate 7/3 (R_{f} 0.6) to give a bright yellow solid (15 mg).

According to Scheme 3, Prop-2-ynyl 4-(Biphenyl-4-yl)-2-ethyl-7,7-dimethyl-5-oxo-1,4,6,6,8,8-hexahydroquinoline-3-carboxylate (BI-3005) may be prepared as follows:

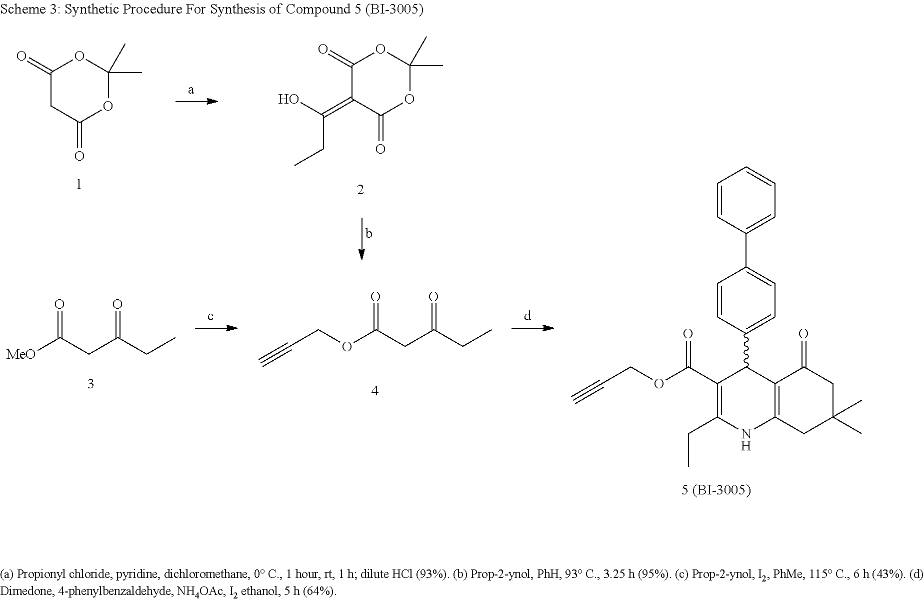

5-(1-Hydroxypropylidene)-2,2-dimethyl-[1,3]dioxane-4,6-dione (2). To a solution of 2,2-dimethyl-1,3-dioxane-4,6-dione (4.41 g, 30.0 mmol) and pyridine (4.85 mL, 60.0 mmol) in dichloromethane (24 mL) at 0° C. under argon is added propionyl chloride (2.95 mL, 33.0 mmol). The mixture is stirred at 0° C. for 1 h and at rt for 1 h before being diluted with 2 N HCl (40 mL) and extracted with dichloromethane (80 mL). The extract is washed (brine) and dried. Solvent is removed at reduced pressure to give 5.57 g (93%) of 2 as a yellow solid, mp 43-46° C. IR 3345, 2856, 1715, 1456 cm⁻; ¹H NMR (CDCl₃) δ 1.29 (t, J=7.5 Hz, 3H, CH₂CH₃), 1.76 (s, 6H, CH₃, CH₃), 3.14 (q, J=7.5 Hz, 2H, CH₂CH₃), 15.42 (bs, 1H, OH).

Prop-2-ynyl 3-Oxopentanoate (4). Method A. A solution of 5-(1-hydroxypropylidene)-2,2-dimethyl-[1,3]dioxane-4,6-dione (2) (2.00 g, 10.0 mmol) and 2-propynol (1.10 g, 20.0 mmol) in benzene (20 mL) is stirred at 93° C. for 3.25 h and then cooled to rt. After solvent removal at reduced pressure, the residue is purified on silica gel (12.5% to 14.3% ethyl acetate/hexane) to give 1.47 g (95%) of 4 as a colorless liquid.

Method B. To a solution of methyl 3-ketopentanoate (3) (822 mg, 6 mmol) and 2-propynol (686 mg, 12.0 mmol) in toluene (7 mL) is added I₂ (46 mg, 0.18 mmol). The mixture is stirred at 115° C. for 6 hours, then cooled to rt, and extracted with ethyl acetate (100 mL). The extract is washed (brine) and dried. After solvent removal at reduced pressure, the residue is distilled (125-130° C., 2 mm) to give 401 mg (43%) of 4 as a colorless liquid. IR 3345, 2856, 1715, 1456 cm⁻¹; ¹H NMR (CDCl₃) δ 1.10 (t, J=7.5 Hz, 3H, CH₂CH₃), 2.52 (t, J=2.4 Hz, 1H, CH≡CCH₂), 2.58 (q, J=7.5 Hz, 2H, CH₂CH₃) 3.51 (s, 2H, CH₂), 4.74 (d, J=2.4 Hz, 2H, CH≡CCH₂).

Prop-2-ynyl 4-(Biphenyl-4-yl)-7,7-dimethyl-2-ethyl-5-oxo-1,4,6,6,8,8-hexahydro-quinoline-3-carboxylate (5, BI-3005). A mixture of prop-2-ynyl 3-oxo-pentanoate (4) (196 mg, 1.27 mmol), 4-phenylbenzaldehyde (244 mg, 1.27 mmol), dimedone (180 mg, 1.27 mmol), ammonium acetate (101 mg, 1.27 mmol), I₂ (97 mg, 0.38 mmol), and ethanol (10 drops) is stirred under argon for 5.5 h and then diluted with 5% Na₂S₂O₃ (30 mL). The resultant suspension is extracted with ethyl acetate (50 mL and 30 mL). The extract is washed (5% Na₂S₂O₃, H₂O, and brine) and dried. After solvent removal at reduced pressure, the residue is crystallized (ethanol) to give 139 mg of 5 (BI-3005) as a cream solid. The residue produced on concentration of the mother liquors is purified on silica gel (16.7% to 66.7% ethyl acetate/hexane) to give an additional 212 mg of 5 (BI-3005) as a cream solid for a total of 0.351 mg (64%), mp 220-222° C. IR 3345, 2856, 1715, 1456 cm⁻¹; ¹H NMR (CDCl₃ IR 3332, 2856, 11693, 1225 cm⁻¹; ¹H NMR (CDCl₃) δ 0.99 (s, 3H, CH3), 1.12 (s, 3H, CH₃), 1.29 (t, J=7.5 Hz, 3H, CH₂CH₃), 2.18-2.44 (m, 5H, COCH₂, CH≡CCH₂, CH₂), 2.77-2.90 (m, 2H, CH₂CH₃), 4.58-4.75 (m, 2H, CH≡CCH₂), 5.16 (s, 1H, CH), 5.87 (bs, 1H, NH), 7.28-7.59 ppm (m, 9H, 4-BiphenylH).

According to Scheme 4, Methyl 7,7-Dimethyl-2-ethyl-5-oxo-4-(4-prop-2-ynyloxy)phenyl)-1,4,6,6,8,8-hexahydroquinoline-3-carboxylate (3, BI-3027) may be prepared as follows:

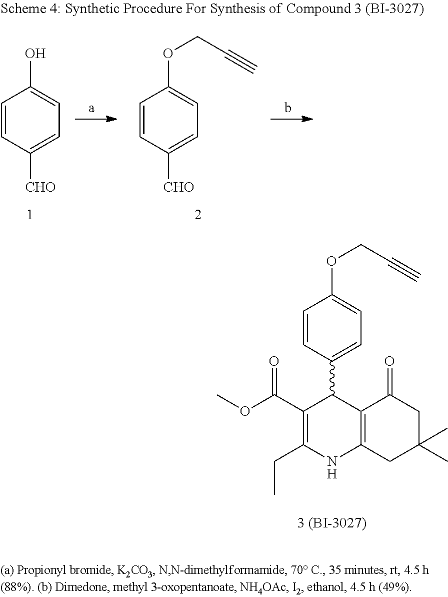

4-(Prop-2-ynyloxy)benzaldehyde (2). (Beena et al. 2009) To a suspension of 4-hydroxybenaldehyde (1) (1.22 g, 10.0 mmol) and K₂CO₃ (4.15 g, 30 mmol) in N,N-dimethylformamide (20 mL) that is stirred at 70° C. under argon for 35 min and then cooled to rt is added 80% prop-2-ynyl bromide (12 mmol) in toluene (1.34 mL). The resulting mixture is stirred for 4.5 hours, quenched with cold H₂O (80 mL), and filtered. The solid is washed with H₂O (2×30 mL) to give 1.41 g (88%) of 2 as a cream solid, mp 80-82° C. IR 2824, 1681, 1250 cm⁻¹; ¹H NMR (CDCl₃) δ 2.60 (t, J=2.4 Hz, 1H, CH≡CCH₂), 4.81, 2H, CH≡CCH₂), 7.12 (d, J=9.3 Hz, 2H, 3,5-ArH), 7.88 (d, J=9.3 Hz, 2H, 2,6-ArH), 9.93 (s, 1H, CHO).

Methyl 7,7-Dimethyl-2-ethyl-5-oxo-4-[(4-prop-2-ynyloxy)phenyl]-1,4,6,6,8,8-hexahydroquinoline-3-carboxylate (3, BI-3027). 4-(Prop-2-ynyloxy)benzaldehyde (2) (203 mg, 1.27 mmol), methyl 3-oxopentanoate (173 mg, 1.27 mmol), dimedone (180 mg, 1.27 mmol), ammonium acetate (101 mg, 1.27 mmol), I₂ (97 mg, 0.38 mmol), and ethanol (10 drops) is stirred under argon for 4.5 hours, then stopped with 5% Na₂S₂O₃ (30 mL), and extracted with ethyl acetate (50 mL and 30 mL). The extract is washed (5% Na₂S₂O₃, H₂O, and brine) and dried. After solvent removal at reduced pressure, the esidue is purified on silica gel (33% to 60% ethyl acetate/hexane) to give 247 mg (49%) of 3 (BI-3027) as a cream solid, mp 162-165° C. IR 3329, 2874, 1684, 1222 cm⁻¹; ¹H NMR (CDCl₃) δ 0.95 (s, 3H, CH₃), 1.10 (s, 3H, CH₃), 1.26 (t, J=7.2 Hz, 311, CH₂CH₃), 2.15-2.41 (m, 4H, COCH₂, CH₂), 2.52 (t, J=2.4 Hz, 1H, CH═CCH₂), 2.76-2.88 (m, 2H, CH₂CH₃), 3.64 (s, 3H, OCH₃), 4.58-4.75 (d, J=2.4 Hz, 2H, CH≡CCH₂), 5.05 (s, 1H, CH), 5.84 (bs, 1H, NH), 6.84 (d, J=8.7 Hz, 2H, 3,5-ArH), 7.24 ppm (d, J=8.7 Hz, 2H, 2,6-ArH).

According to Scheme 5, Methyl 7,7-Dimethyl-2-ethyl-4-(4-ethynylphenyl)-5-oxo-1,4,6,6,8,8-hexahydroquinoline-3-carboxylate (2, BI-3029) and Methyl 7,7-Dimethyl-2-ethyl-5-oxo-4-[4-(1-phenyl[1,2,3]triazol-4-yl)phenyl]-1,4,6,6,8,8-hexahydroquinoline-3-carboxylate (3, BI-3041) may be prepared as follows:

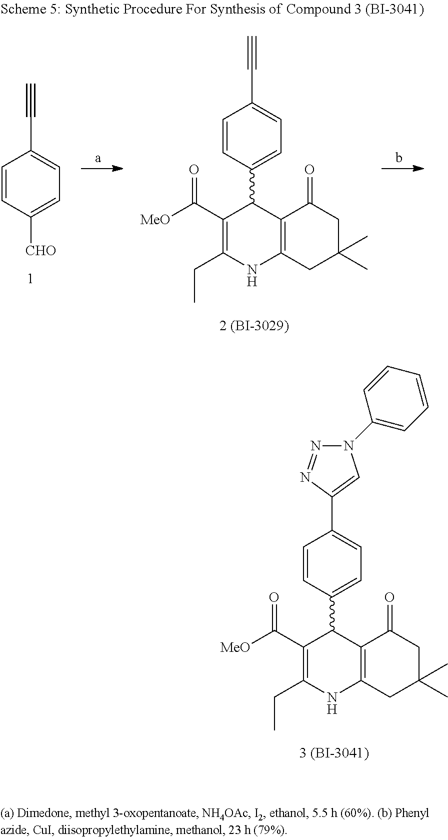

Methyl 7,7-Dimethyl-2-ethyl-4-(4-ethynylphenyl)-5-oxo-1,4,6,6,8,8-hexahydroquinoline-3-carboxylate (2, BI-3029). A mixture of 4-ethynylbenzaldehyde (1) (171 mg, 1.27 mmol), methyl 3-oxopentanoate (175 mg, 1.27 mmol), dimedone (181 mg, 1.27 mmol), ammonium acetate (101 mg, 1.27 mmol), I₂ (97 mg, 0.38 mmol), and ethanol (10 drops) is stirred under argon for 5.5 hours, quenched with 5% Na₂S₂O₃ (30 mL), and extracted with ethyl acetate (50 mL and 30 mL). The extract is washed (5% Na₂S₂O₃, H₂O, and brine) and dried. After solvent removal at reduced pressure, the residue is purified on silica gel (33% to 60% ethyl acetate/hexane) to give 277 mg (60%) of 2 (BI-3029) as a cream solid, mp 234-236° C. IR 3332, 2933, 1704, 1610, 1482, 1214 cm⁻¹; ¹H NMR (CDCl₃) δ 0.93 (s, 3H, CH₃), 1.10 (s, 3H, CH₃), 1.26 (t, J=7.2 Hz, 3H, CH₂CH₃), 2.07-2.42 (m, 4H, COCH₂, CH₂), 2.76-2.89 (m, 2H, CH₂CH₃), 3.02 (s, 1H, CH≡C), 3.63 (s, 3H, OCH₃), 5.09 (s, 1H, CH), 5.90 (bs, 1H, NH), 7.27 (d, J=8.1 Hz, 2H, 3,5-ArH), 7.37 ppm (d, J=8.1 Hz, 2H, 2,6-ArH).

Methyl 7,7-Dimethyl-2-ethyl-5-oxo-4-[4-(1-phenyl[1,2,3]triazol-4-yl)phenyl]-1,4,6,6,8,8-hexahydroquinoline-3-carboxylate (3, BI-3041). A suspension of methyl 7,7-dimethyl-2-ethyl-4-(4-ethynylphenyl)-5-oxo-1,4,6,6,8,8-hexahydroquinoline-3-carboxylate (2, BI-3029) (22 mg, 0.06 mmol), phenyl azide (7 mg, 0.06 mmol), CuI (4.6 mg, 0.024 mmol), and diisopropylethylamine (105 μL, 0.6 mmol) in methanol (2.4 mL) and tetrahydrofuran (0.5 mL) is stirred for 23 h and then concentrated at reduced pressure. The residue is purified on silica gel (50% to 60% ethyl acetate/hexane) to give 23 mg (79%) of 3 (BI-3041) as a cream solid, mp 238-240° C. IR 3300, 2966, 1689, 1608, 1489, 1215 cm⁻¹; NMR (CDCl₃) δ 0.81 (s, 3H, CH₃), 1.02 (s, 3H, CH₃), 1.21 (t, J=7.5 Hz, 3H, CH₂CH₃), 2.07-2.46 (m, 4H, COCH₂, CH₂), 2.71-2.91 (m, 2H, CH₂CH₃), 3.65 (s, 3H, OCH₃), 5.14 (s, 1H, CH), 7.47-7.62 (m, 5H, PhH), 7.80 (d, J=7.2 Hz, 2H, 3,5-ArH), 7.83 (d, J=7.2 Hz, 2H, 2,6-ArH), 8.22 (s, 1H, triazH), 8.25 ppm (bs, 1H, NH).

According to Scheme 6, Prop-2-ynyl 4-(Biphenyl-4-yl)-7,7-dimethyl-2-ethyl-5-oxo-1,4,6,6,8,8-hexahydroquinoline-3-carboxylate (3, BI-3036) may be prepared as follows:

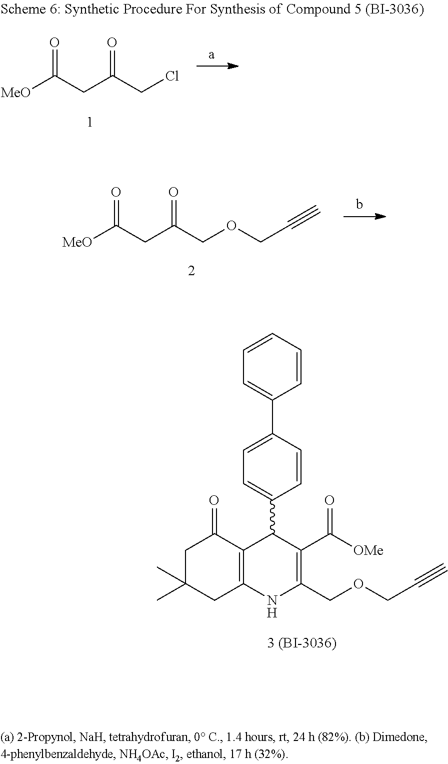

Methyl 3-Oxo-4-(prop-2-ynyloxy)butyrate (2). To a solution of methyl 4-chloroacethyl acetateetate (1.2 g, 8.0 mmol) in tetrahydrofuran (12 mL) at 0° C. under argon is added 60% NaH (16 mmol) in mineral oil (640 mg) followed by 2-propynol (448 mg, 8 mmol). This suspension is stirred at 0° C. for 1.4 h and at rt for 24 h before dilution with cold 2 N HCl (25 mL) and extraction with diethyl ether (50 mL and 2×40 mL). The extract is washed (saturated NaHCO₃ and brine) and dried. After solvent removal at reduced pressure, the residue is purified on silica gel (20% to 25% ethyl acetate/hexane) to give 987 mg (82%) of 2 as a light-yellow liquid. IR 2960, 1747, 1722, 1328, 1098 cm⁻¹; ¹H NMR (CDCl₃) 2.53 (t, J=2.4 Hz, 1H, CH≡CH₂), 3.60 (s, 2H, COCH₂CO), 3.78 (s, 3H, OCH₃), 4.28 (s, 2H, COCH₂O), 4.30 ppm (d, J=2.4 Hz, 2H, CH≡CCH₂).

Prop-2-ynyl 4-(Biphenyl-4-yl)-7,7-dimethyl-2-ethyl-5-oxo-1,4,6,6,8,8-hexahydro-quinoline-3-carboxylate (3, BI-3036). A mixture of methyl 3-oxo-4-(prop-2-ynyloxy)butyrate (2) (220 mg, 1.29 mmol), 4-phenylbenzaldehyde (247 mg, 1.29 mmol), dimedone (183 mg, 1.29 mmol), ammonium acetate (103 mg, 1.29 mmol), I₂ (98 mg, 0.39 mmol), and ethanol (15 drops) is stirred under argon for 17 hours, quenched with 5% Na₂S₂O₃ (30 mL), and extracted with ethyl acetate (50 mL and 30 mL). The extract is washed (5% Na₂S₂O₃, H₂O, and brine), and dried. After solvent removal at reduced pressure, the residue is crystallized (ethanol) to give 190 mg (32%) of 3 (BI-3036) as a cream solid, mp 204-205° C. IR 3370, 2949, 1691, 1638, 1469, 1216 cm⁻¹; NMR (CDCl₃) δ 0.99 (s, 3H, CH₃), 1.12 (s, 3H, CH₃), 2.19-2.48 (m, 4H, COCH₂, CH₂), 2.56 (t, J=2.4 Hz, 1H, CH≡CCH₂), 3.67 (s, 3H, OCH₃), 4.34 (d, J=2.4 Hz, 2H, CH≡CCH₂), 4.86-4.98 (m, 2H, CH₂O), 5.14 (s, 1H, CH), 7.15 (bs, 1H, NH), 7.30-7.60 ppm (m, 9H, 4-BiphenylH).

### Example 3

### Activity of Compounds of General Structure I

The potency of several compounds of the above shown general structure I using the above-described testing methods is measured. Approximately 150 dihydropydirine diones analogs were synthesized. Table 1-1 provides the activity of some of the compounds of structure IA and IB in the cardiomyocyte screening assay.

TABLE 1-1

| | Salt | | | Salt | |
|---|---|---|---|---|---|
| STRUCTURE^{a} | Form | Activity^{b} | STRUCTURE | Form | Activity |
| 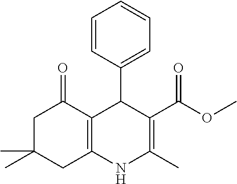 | none | ++ | 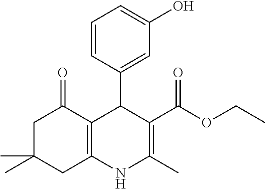 | none | + |
| 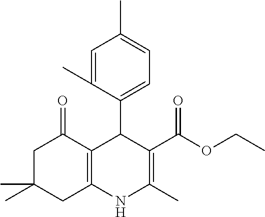 | none | + | 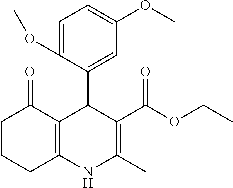 | none | + |
| 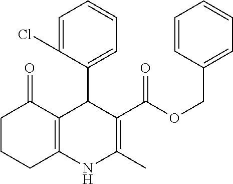 | none | + | 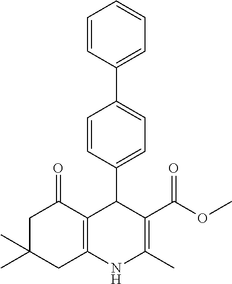 | none | +++ |
| 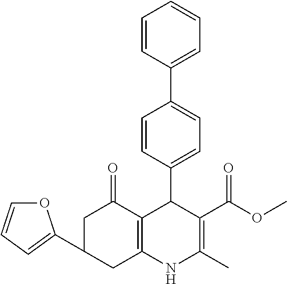 | none | ++ | 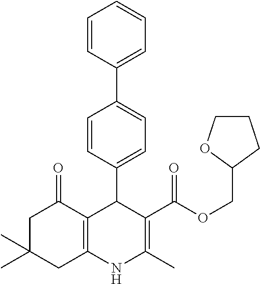 | None | ++ |
| 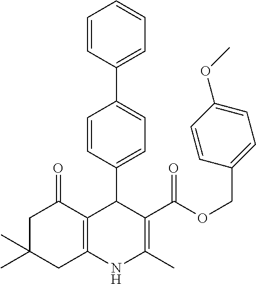 | None | +++ | 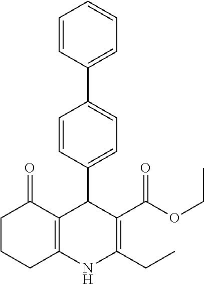 | None | + |
| 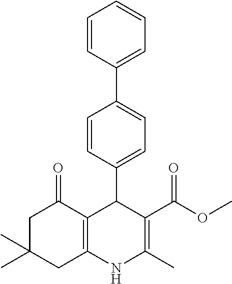 | Oxalate | + | 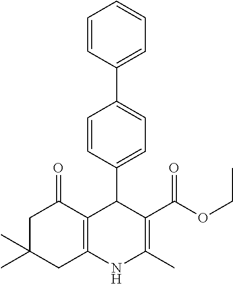 | None | + |
| 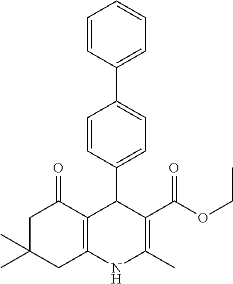 | HCl | +++ | 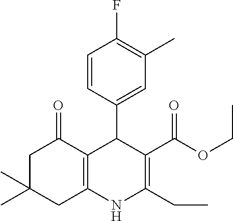 | HCl | + |
| 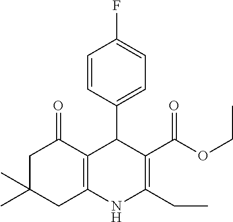 | None | + | 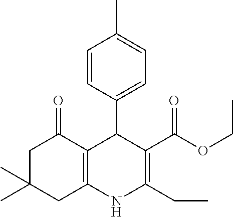 | HCl | + |
| 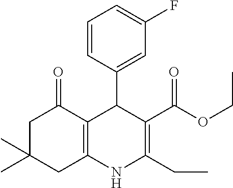 | HCl | + | 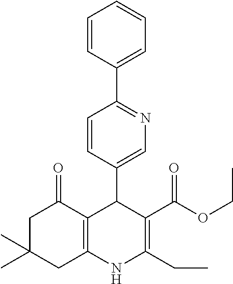 | HCl | ++ |
| 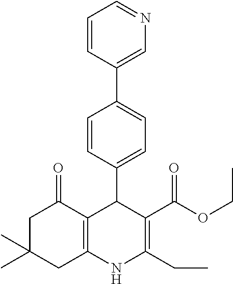 | HCl | ++ | 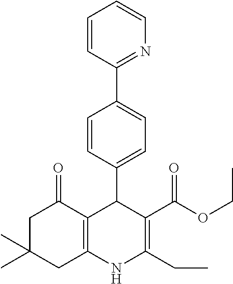 | n | ++ |
| 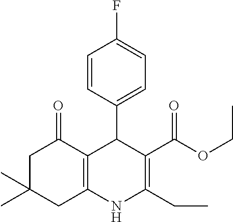 | HCl | ++++ | 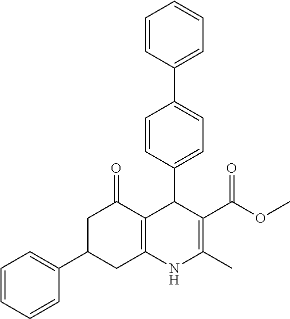 | HCl | NA |
| 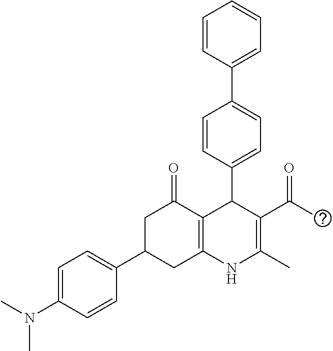 | HCl | NA | 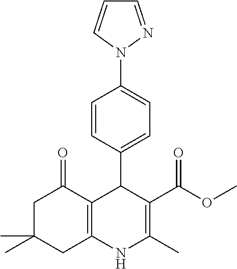 | HCl | NA |
| 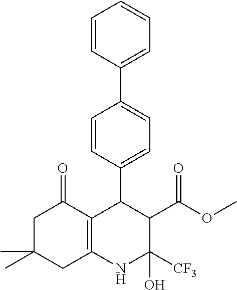 | HCl | NA | 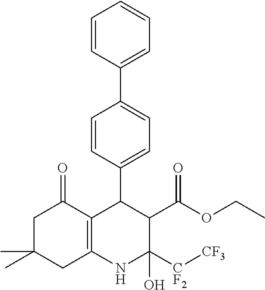 | none | NA |
| 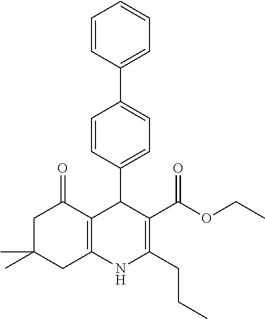 | HCl | NA | 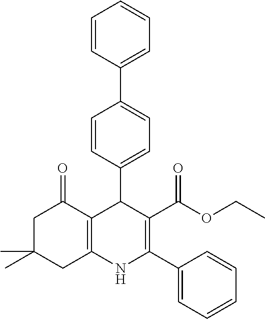 | none | NA |
| 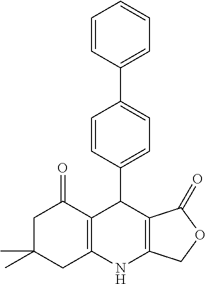 | HCl | NA | 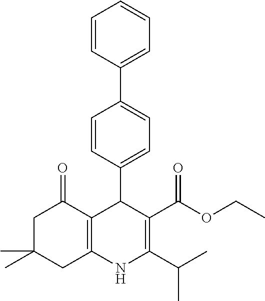 | none | NA |

^{a}All the compounds have been characterized by LRMS and/or ¹H NMR.
^{b}Activity is based on compound 22 being 100% activity; ++++: >80% activity compared to 22; +++ between 60 to 80% activity compared to 2; ++ between 40 to 60% activity compared to 2; +: <40% activity compared to compound 22. NA, not available.

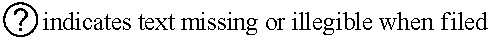

Table 1-2 provides the characterization of compounds of structure IA and IB. Nuclear magnetic resonance data were recorded on a Varian Mercury 300 MHz Spectrometer using TMS as the internal standard and CDCl₃ as the solvent except where indicated. Electrospray mass spectral (MS) data was obtained using a Hitachi M-7000.

TABLE 1-2

| | MW | |
|---|---|---|
| Structure | (g•mol−¹) | M(1 + H) |
| 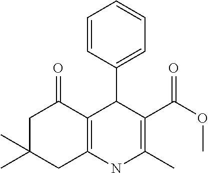 | 325.3 | MS (ESI⁺) m/z 326 (M + H)⁺ |
| 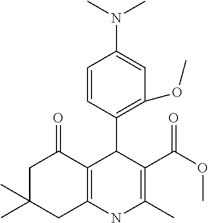 | 398.4 | MS (ESI⁺) m/z 399 (M + H)⁺ |
| 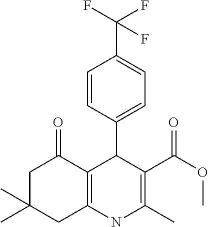 | 393.4 | MS (ESI⁺) m/z 394 (M + H)⁺ |
| 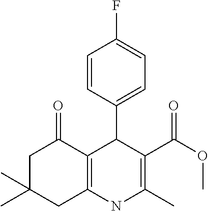 | 343.4 | MS (ESI⁺) m/z 344 (M + H)⁺ |
| 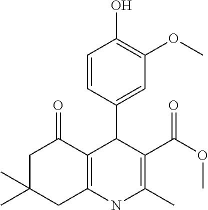 | 371.4 | MS (ESI⁺) m/z 372 (M + H)⁺ |
| 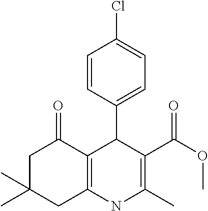 | 359.8 | MS (ESI⁺) m/z 361 (M + H)⁺ |
| 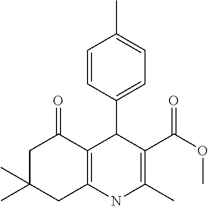 | 339.4 | MS (ESI⁺) m/z 340 (M + H)⁺ |
| 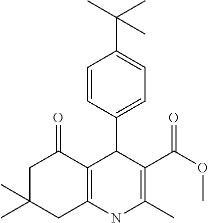 | 381.5 | MS (ESI⁺) m/z 382 (M + H)⁺ |
| 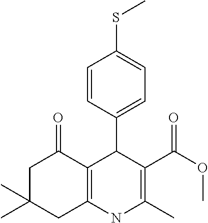 | 371.5 | MS (ESI⁺) m/z 372 (M + H)⁺ |
| 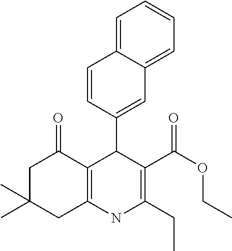 | 403.5 | MS (ESI⁺) m/z 404 (M + H)⁺ |
| 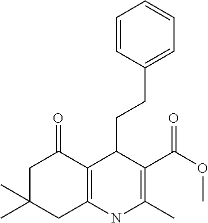 | 353.5 | MS (ESI⁺) m/z 355 (M + H)⁺ |
| 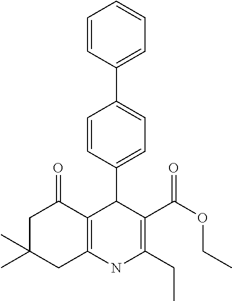 | 429.5 | MS (ESI⁺) m/z 452 (M + Na)⁺ |
| 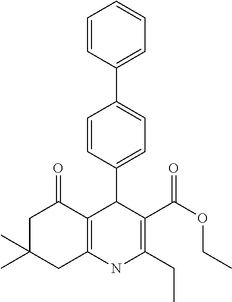 | 466 | MS (ESI⁺) m/z 452 (M + Na)⁺ |
| 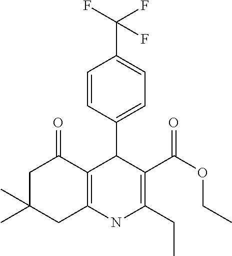 | 422.4 | MS (ESI⁺) m/z 423 (M + H)⁺ |
| 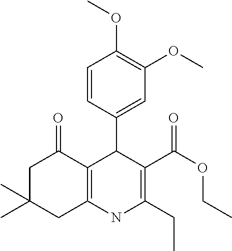 | 413.5 | MS (ESI⁺) m/z 436 (M + Na)⁺ |
| 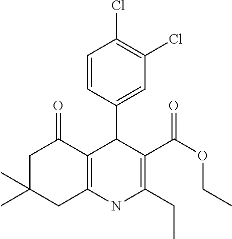 | 422 | MS (ESI⁺) m/z 445 (M + Na)⁺ |
| 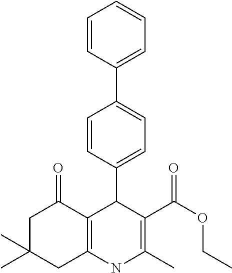 | 415 | MS (ESI⁺) m/z 438 (M + Na)⁺ |
| 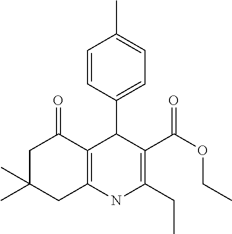 | 367.5 | MS (ESI⁺) m/z 390 (M + H)⁺ |
| 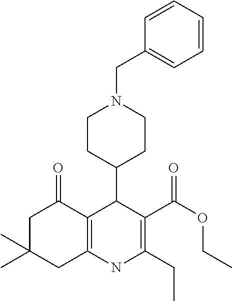 | 450.3 | MS (ESI⁺) m/z 451 (M + H)⁺ |
| 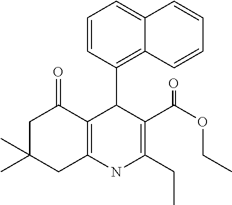 | 403.5 | MS (ESI⁺) m/z 426 (M + Na)⁺ |
| 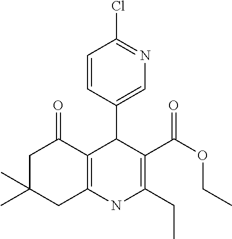 | 389 | MS (ESI⁺) m/z 389.9 (M + H)⁺ |
| 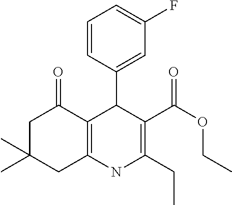 | 371.2 | MS (ESI⁺) m/z 394 (M + Na)⁺ |
| 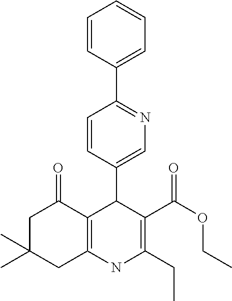 | 430 | MS (ESI⁺) m/z 431 (M + H)⁺ |
| 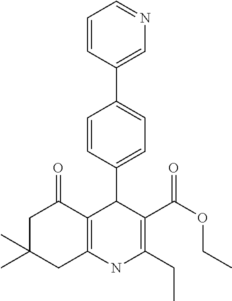 | 430 | MS (ESI⁺) m/z 432 (M + H)⁺ |
| 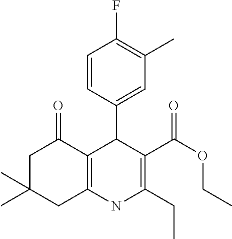 | 385.5 | MS (ESI⁺) m/z 387 (M + H)⁺ |
| 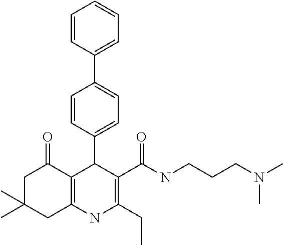 | 485.7 | MS (ESI⁺) m/z 486 (M + H)⁺ |
| 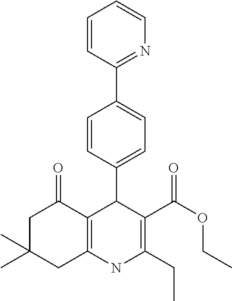 | 430 | MS (ESI⁺) m/z 453.5 (M + H)⁺ |
| 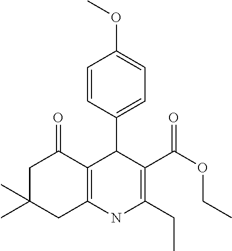 | 383 | MS (ESI⁺) m/z 406.6 (M + H)⁺ |
| 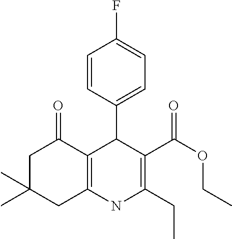 | 371 | MS (ESI⁺) m/z 372 (M + H)⁺ |
| 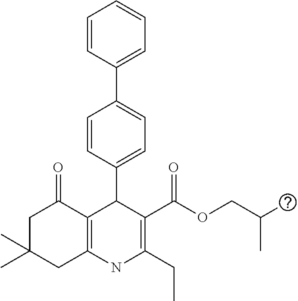 | 457.6 | MS (ESI⁺) m/z 458 (M + H)⁺ |
| 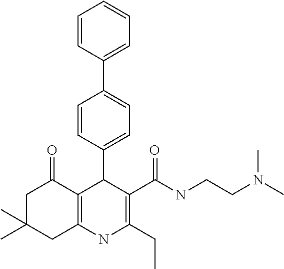 | 471.6 | MS (ESI⁺) m/z 472 (M + H)⁺ |
| 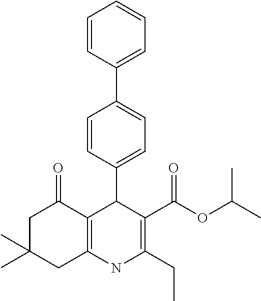 | 443.6 | MS (ESI⁺) m/z 446 (M + H)⁺ |
| 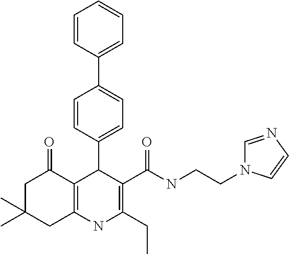 | 494.6 | MS (ESI⁺) m/z 495.7 (M + H)⁺ |
| 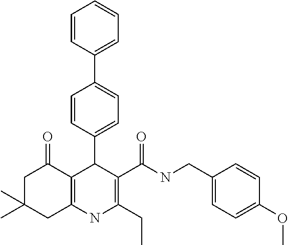 | 520.7 | MS (ESI⁺) m/z 519.3 (M − H)⁺ |

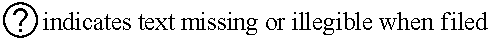

As may be observed from the data presented in Table 1-1, both free bases and salts gave significant potency. Solubility may be a significant factor in the bioactivity of the compounds. On the basis of the lipophilic character of many of the “hits,” it is likely interaction site on the molecular target has lipophilic character. Table 1-3 provides the activity of the compounds of Formula I in the cardiomyocyte screening assay.

TABLE 1-3

| | | | Rel | Rel | Rel |
|---|---|---|---|---|---|
| | Raw GFP | Raw GFP | DMSO | DMSO | DMSO |
| Compound | 0.33 μM | 0.66 μM | 0.33 μM | 0.66 μM | Sum/2 |
| 22 | 2587614 | 979637.6 | 7.715564 | 2.995879 | 5.355722 |
| 20 | 1598064 | 822670.7 | 4.764995 | 2.515851 | 3.640423 |
| 15 | 186829.8 | 1834100 | 0.557076 | 5.608955 | 3.083015 |
| 17 | 1723650 | 168840.5 | 5.139459 | 0.51634 | 2.827899 |
| 18 | 391177.2 | 1396007 | 1.166385 | 4.269199 | 2.717792 |
| 19 | 298951.8 | 1205577 | 0.891393 | 3.686837 | 2.289115 |
| 16 | 346366.7 | 976547.3 | 1.032772 | 2.986429 | 2.0096 |
| 21 | 287436.9 | 943856.5 | 0.857059 | 2.886455 | 1.871757 |

### Example 4

### Activity of Compounds of Formula I

A high content screen (HCS) assay was developed for discovery of cardiomyocyte differentiation agents using a CGR8 mouse embryonic stem cell (ESC) line that had been engineered to express eGFP under control of the α-myosin heavy chain (αMHC) promoter. Maturing cardiomyocytes were identified by expression of contractile proteins such as αMHC as soon as 8 days after the initiation of differentiation. At day 4 after the mESC were separated from the SC growing support (mouse embryonic fibroblast) compounds were administered to the cells. The assay was terminated at day 10. An automated microscope was used to identify molecules able to stimulate differentiation based on phenotype and fluorescence intensity. Differentiation activities were expressed relative to those of the vehicle control as fold-increases in fluorescence and then normalized to the fold-response of DMSO. Screen was conducted at two concentrations (0.33 and 0.66 μM). Table 1-4 provides a summary of effect of 15-22 on cardiomyocyte differentiation from mouse stem cells.

TABLE 1-4

| | *Rel DMSO | *Rel DMSO |
|---|---|---|
| Compound | 0.33 μM | 0.66 μM |
| 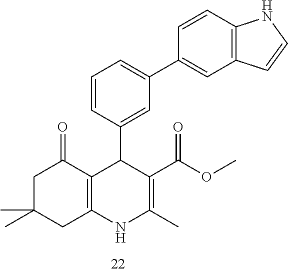 | 7.7 | 2.99 |
| 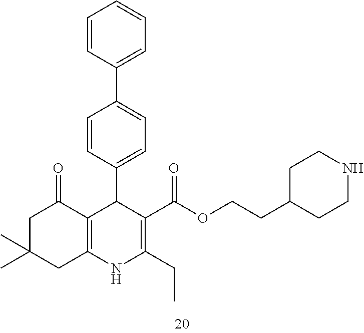 | 4.76 | 2.51 |
| 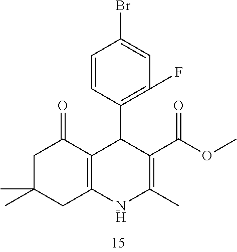 | 0.56 | 5.61 |
| 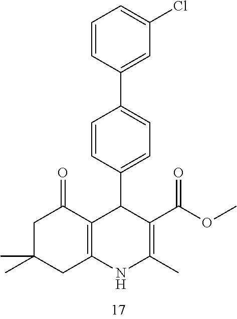 | 5.14 | 0.51 |
| 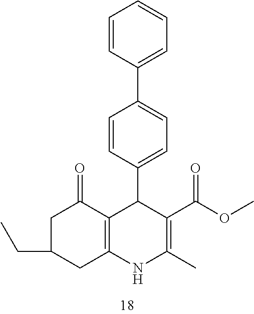 | 1.17 | 4.27 |
| 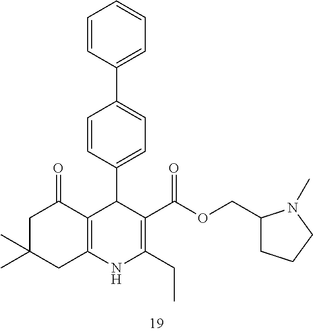 | 0.89 | 3.69 |
| 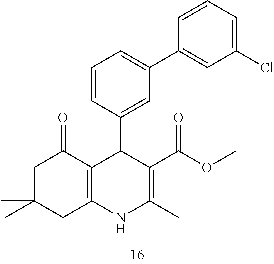 | 1.03 | 2.98 |
| 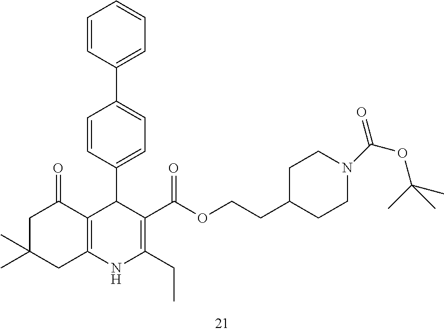 | 0.86 | 2.88 |

*The effect is reported as fold-increase in cardiogenesis compared to DMSO at two different concentrations (0.33 and 0.66 μM).

### Example 5

### General Synthetic Procedure for Obtaining Compounds of Structure II

The benzimidazole-based compounds of general structure II:

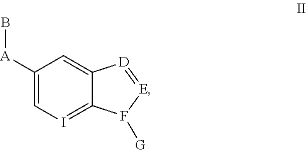

may be synthesized according to the following schemes (when D is nitrogen or when D is carbon).

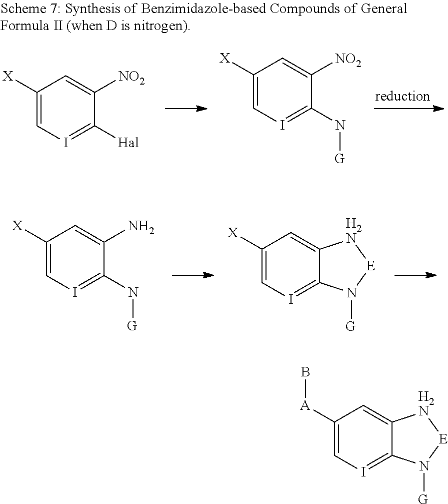

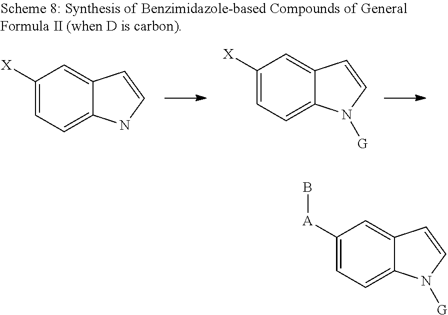

Approximately 600 benzimidazole analogues were synthesized and screened for their ability to facilitate cardiomyocyte differentiation. Analogue solubility is an issue during early phases of screening. Therefore, in addition to the free bases, the salts (i.e., hydrochloride or mono-oxalate) of the target compounds were generated and tested.

Generally, the salts were more soluble under aqueous conditions than the corresponding free bases. In some cases, an EC₅₀ value for a free base could not be determined because of compound precipitation from the medium; however, evaluation of the corresponding salt provided the EC₅₀ value. Compound library generation is then modified so that hydrochloride salts of the substituted benzimidazoles were isolated from the synthesis. In addition to ease of synthesis, the increased solubility of the benzimidazole salts appeared necessary for potency.

The phenothiazine-based compounds of general structure III, IV and V:

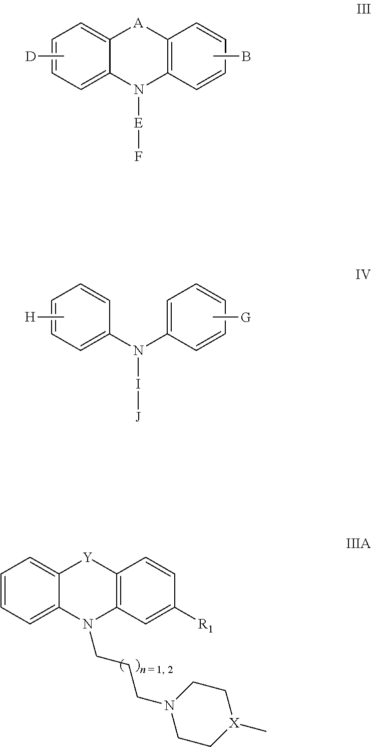

may be synthesized according to the following scheme.

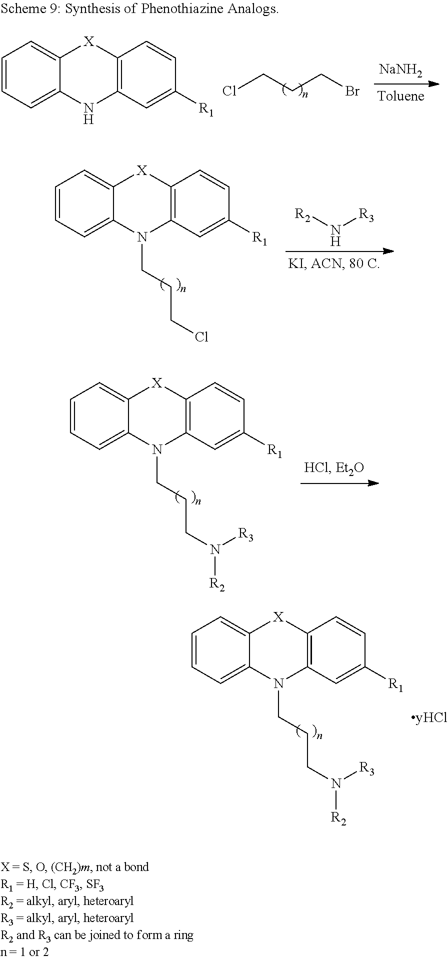

Activity of Compounds of General Structures I, II and III

The potencies (i.e., the EC₅₀ values) of the compounds of the above shown general structure I and III using the above-described testing methods is measured. Table 3-1 provides the activity of compounds of structure I and III with potencies in the cardiomyocyte screening assay.

### Example 6

### General Synthetic Procedure for Obtaining Compounds of Structure V, VI, andD VII

The tamoxifen-based compounds of structure V, VI and VII:

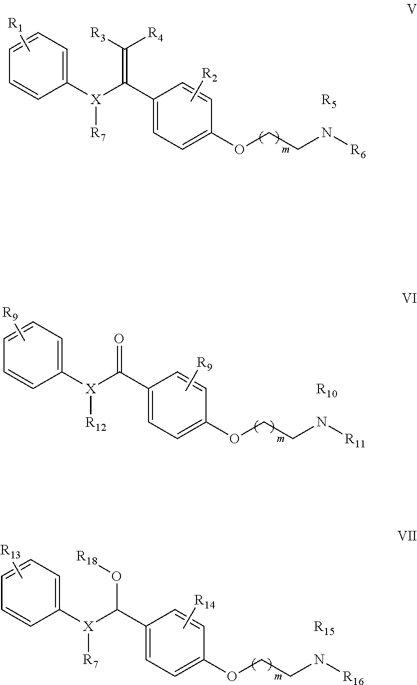

may be synthesized according to the following schemes.

The compounds of the above shown general structure VI and VII may be synthesized according to the following schemes.

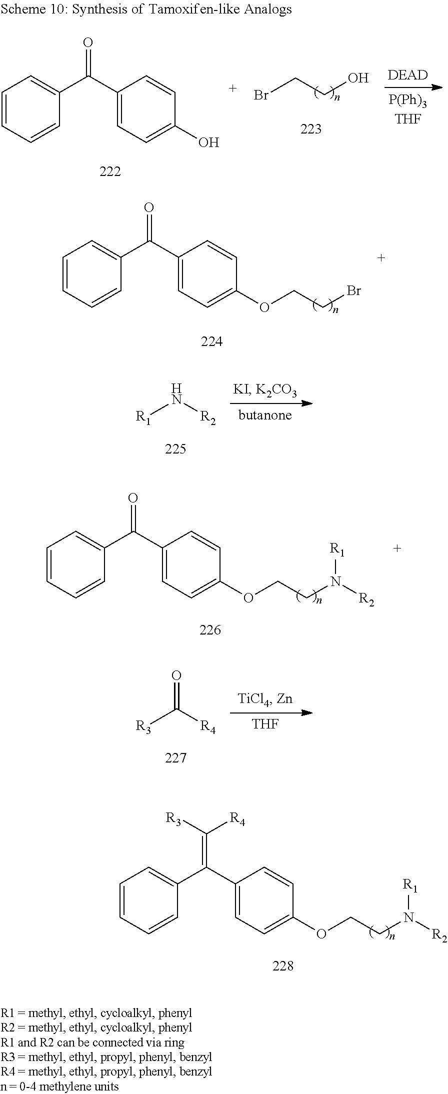

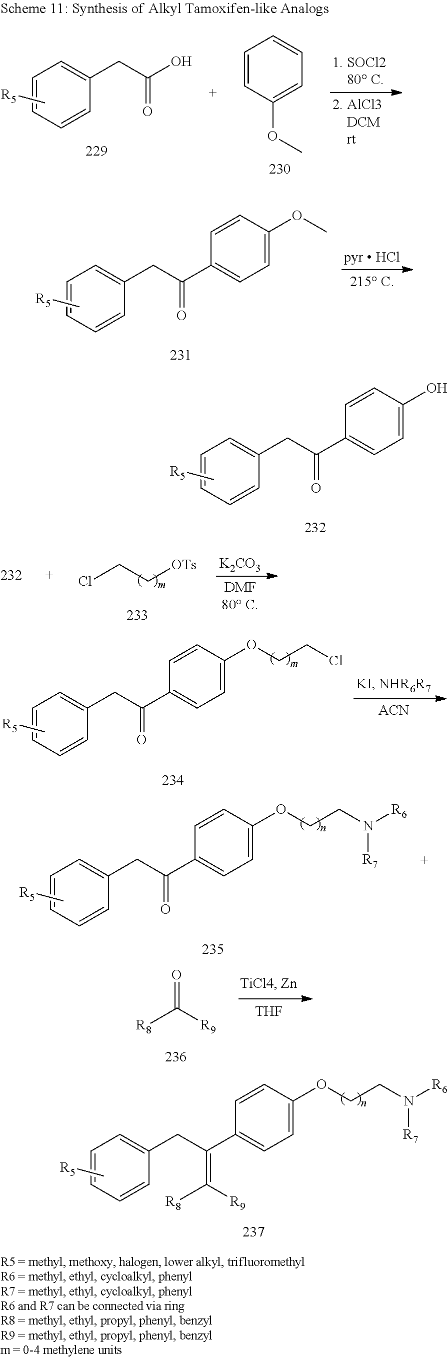

### Example 7

### Study of Small Molecule Inducers of Stem Cell Cardiogenesis

A mouse embryonic stem cell (mESC)-based high throughput assay used to screen a commercially available and diverse small molecule library to identify small molecules that stimulate cardiomyocyte differentiation. The assay is developed to probe compounds that act between 2 and 6 days of differentiation in monolayer culture, corresponding to the time window when the ESCs become specified to follow the cardiomyocyte lineage. The assay readout is eGFP expression from the cardiomyocyte-specific alpha myosin heavy chain (aMHC) gene. eGFP fluorescence is imaged by high throughput microscopy (HTM) and quantified by calculating the integrated fluorescence intensity within intensity thresholded mask of areas of cardiomyocyte differentiation. About 30,000 data points were screened encompassing ˜14,000 unique small molecules, each tested at 1 and 5 μg/mL doses. After data analysis and filtration of artifacts using statistics and visual confirmation in images, 14 compounds were reordered and verified with a secondary confirmation screen. Of the potential hits, 3 compounds with strong cardiogenic potential are described below.

A biological time course experiment suggested the biological action of each molecule is maximized at overlapping but non-identical developmental windows between days 2 to 5 of mESC to cardiomyocyte differentiation. Early analysis of molecular markers induced in secondary assays suggest that these compounds act by regulating mesoderm and endodermal patterning, consistent with the time frame when they are active. An SAR effort is undertaken to investigate the structure-activity relationship (SAR) of all 3 “hit” molecules with the goal of identifying an optimized structure yielding maximum biological potency; and molecular space amenable to affinity ligand linkage without abrogating biological activity. The medicinal chemistry and SAR studies for 1) benzimidazole, 2) dihyropyridine and 3) phenothiazine classes are described.

The molecules would be expected to be used for stimulating differentiation of stem cell cells, in particular but not limited to embryonic stem cells (ESCs) and induced pluripotent stem cells (IPSCs) to endoderm (e.g., liver, lung and pancreas) and cardiac derivatives.

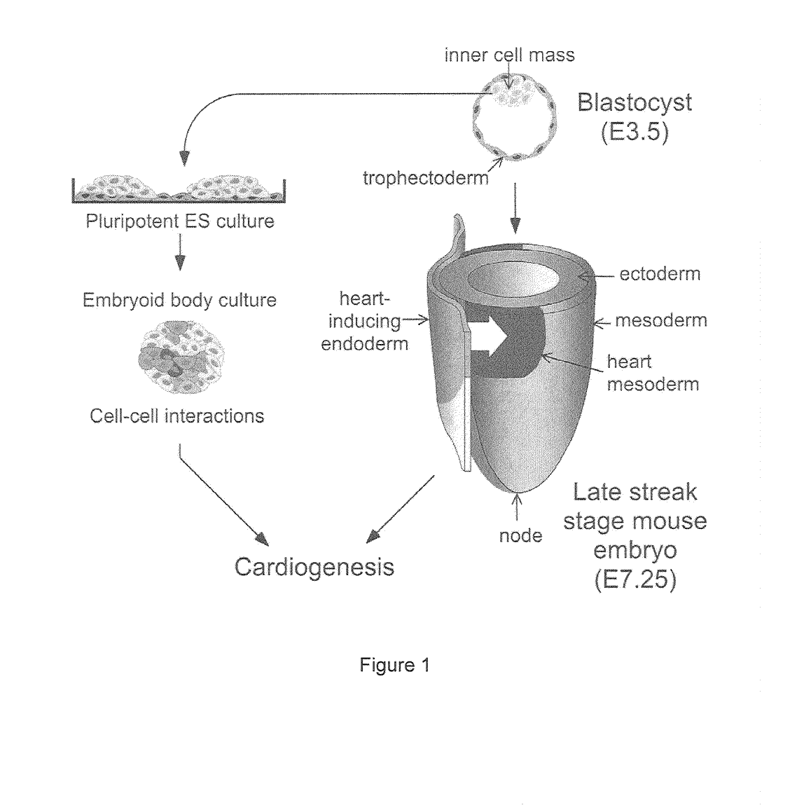

Tissue recombination assays were used leading to the identification of non-cardiac mesoderm and endoderm as sources of heart-inducing factors. The results are demonstrated by FIG. 1 (for mouse), demonstrating comparison of heart induction in mouse embryos and mESCs. ESCs induced to differentiate by aggregation into embryoid bodies (EBs) form all three germ layers (ectoderm, mesoderm and endoderm) then spontaneously develop a small number of cardiomyocytes, probably by preserving cellular interactions that occur in normal embryogenesis.

As can be seen from the information shown by FIG. 1, mESCs are derived from the inner cell mass of pre-implantation embryos (˜E3.5, top). Heart induction in EBs probably recapitulates cell-cell interactions in early embryo, in which anterior visceral and definitive endoderm initiates cardiogenesis within the adjacent heart-forming mesoderm (dark red). Most of the endoderm (yellow) in this diagram is shown peeled away. The heart-inducing region (grey) consists of the extra-embryonic anterior visceral endoderm and anterior definitive endoderm.

### Example 8

### Study of Natural Protein Inducers

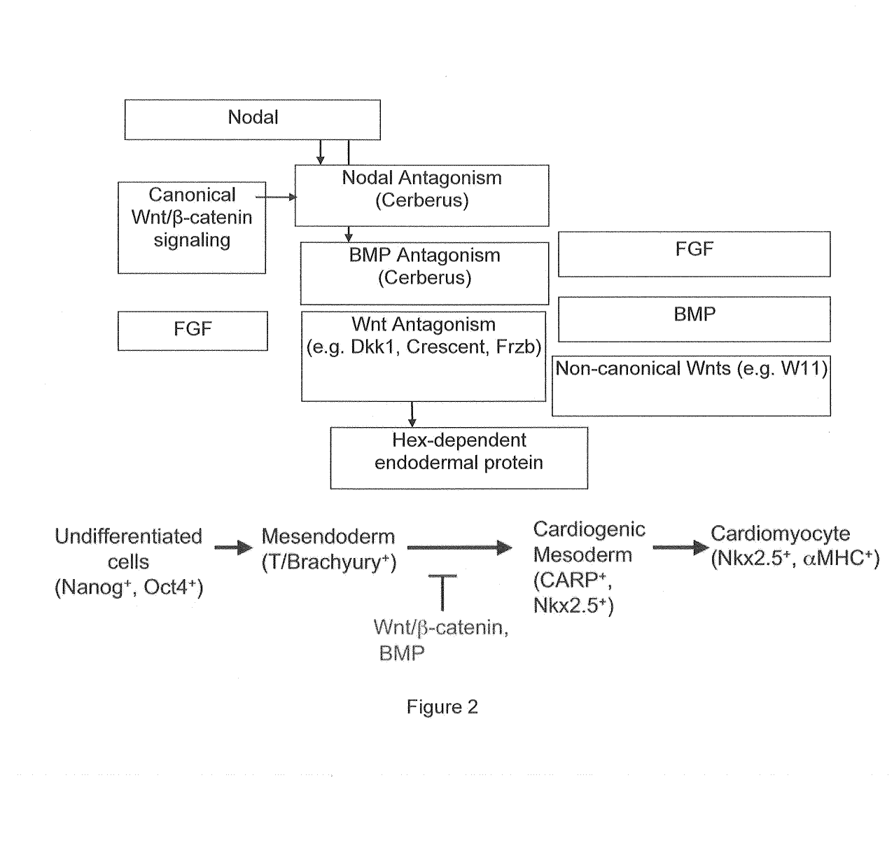

Natural proteins that induce heart tissue in embryos operate in temporally complex patterns so that some factors act early, then are repressed and later re-activated, as shown by FIG. 2 which provides summary model for signaling pathways in cardiomyocyte formation and demonstrates the dynamics of secreted pathway activators functioning alternately with pathway antagonists during the developmental progression from stem cells to cardiomyocytes. In parentheses are the gene promoters to be used in the proposed research to mark the discrete stages of cardiomyogenesis.

It has been previously reported that fibroblast growth factor (FGF), Wnt and Nodal are essential for mesendoderm formation (mammalian streak tissue) along the anteroposterior body axis. Wnt antagonists, particularly Dickkopf 1 (Dkk) 1, are involved in patterning anterior mesendoderm and initiate cardiogenesis by activating the homeodomain protein Hex. Cardiogenesis is enhanced by activation of non-canonical (non-β-catenin) Wnt signaling pathways. Canonical Wnt signaling acts early in both ESCs and embryos, whereas its inhibition appears to occur later. TGF β-family member Nodal and its co-receptor Cripto also induce heart cells in embryos and mESCs. BMPs via Smad transcription factors promote cardiogenesis in embryos and synergize with FGF isoforms to extend the cardiogenic region posteriorly. Embryological studies in Xenopus suggest that BMPs function after Wnt antagonism or Nodal to sustain cardiogenesis from the NRx2.5+ state onward. Findings that BMPs also stimulate cardiogenesis of ESCs and adult heart ScaI+ cells support the concept that factors operating during embryogenesis can stimulate ESC and potentially adult stem cell cardiomyogenesis.

There have been a few published screens for small molecule inducers of ESC cardiogenesis. These include a very small screen of a few hundred compounds that identified ascorbic acid, a larger screen that identified a few compounds that were named cardiogenols, an earlier hit from the screen described in this application that resembled PPAR agonists but did not activate any PPAR, and a recent screen that identified a sulfonamide compound. The latter compound appears to act earlier than the disclosure compounds.

### Example 9

### Study of Activities of Disclosure Compounds

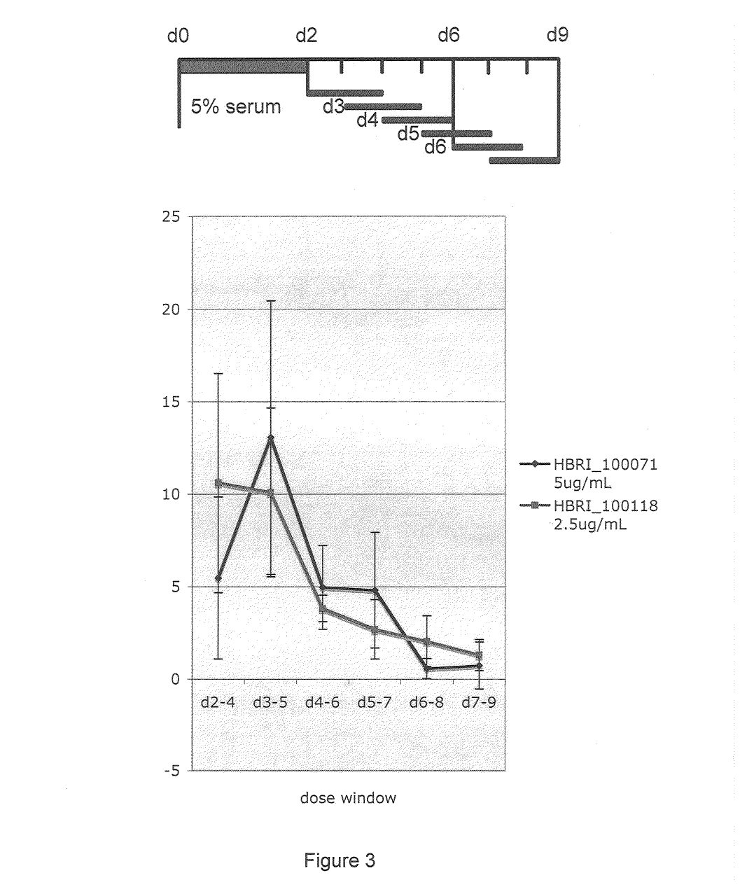

Compounds were examined for activity potency in different 2 day windows, spanning 2-4, 3-5, 4-6, 5-7 and 6-8 days of differentiation. FIG. 3 shows that the compounds are active between days 2-4 and 3-5, but not thereafter.

The time frame of 2-5 days is consistent with action after the primary mesoderm is induced and when this tissue is specified to differentiate into the particular types of mesoderm and endoderm that form in the cultures. This process is termed mesoderm and endoderm patterning and is essential to produce the correct types of mesoderm and endoderm for further differentiation. The heart, as well as adjacent tissues of the head, pancreas, liver, lung, thymus, among other tissues, form from anterior mesoderm and endoderm specified at this time.

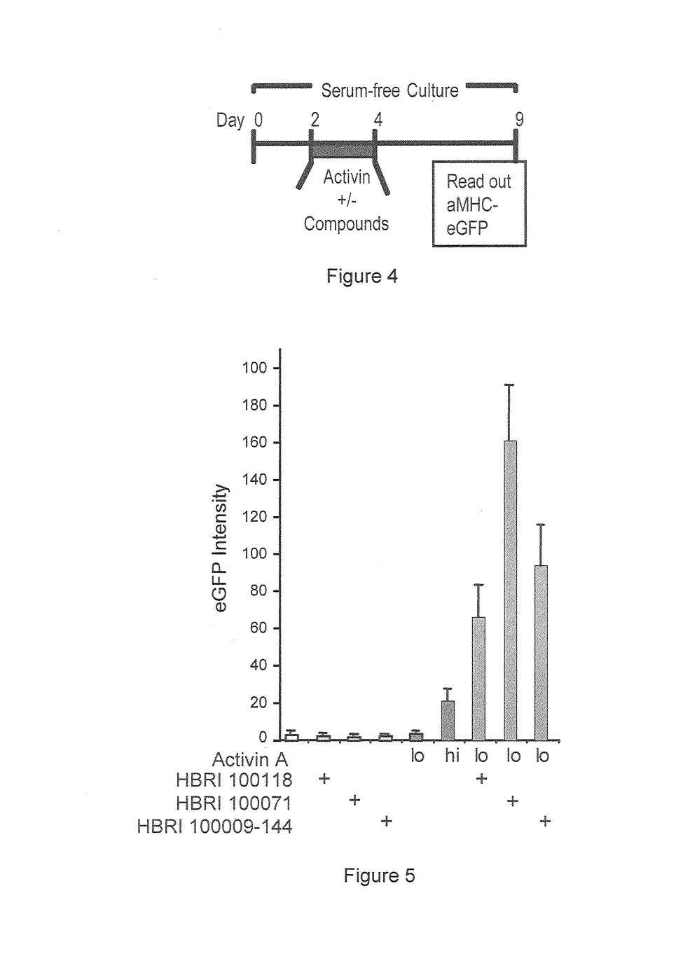

To define the point of action in more detail, a more focused assay is designed to probe the activities of the compounds between days 2-4 of differentiation. Of note is that the original assay is performed in the presence of serum, which activates many pathways that obscure the targets of the small molecules. Since serum activates many pathways and many genes, its presence confounds analysis of downstream targets. Thus, a serum-free mESC cardiac differentiation assay is set up to more precisely validate the activity of the compounds and determine if they acted independently or synergized with a known inducer. The refined assay, diagrammed in FIG. 4, tested the ability of the small molecules to stimulate mesodermal differentiation from day 2 to day 4. Differentiation is initiated by aggregating mESC into embryoid bodies (EBs) in serum free conditions. Day 2 EBs were dispersed in the presence of growth factors or small molecules to allow maximal exposure. Cells were allowed to re-aggregate into EBs until day 4 when they were dispersed and plated in conducive conditions for cardiac differentiation to day 9, at which point eGFP is imaged and quantified by the algorithm described above. The conducive environment in some experiments is to plate the treated stem cells onto another cell line, END2, and in other experiments is to plate the treated stem cells onto fibronectin in the presence of other growth factors, including FGF2, because both provide a permissive environment for cardiogenic mesoderm to develop to cardiomyocytes but do not induce cardiomyocytes.

FIG. 5 summarizes experiments showing synergy with Activin/Nodal signaling. Exposure to high Activin A, an inducer of anterior mesoderm and endoderm, in the day 2 to day 4 window of this assay results in efficient cardiogenesis, whereas treatment with posteriorizing growth factors such as BMP4 does not. When the small molecules from the three classes alone are added in this time frame, no activity is observed, suggesting that they do not themselves mimic Activin/Nodal signaling but “synergize” with this pathway. Indeed, when the small molecules were added in presence of low doses of Activin A from day 2 to day 4, an increased amount of cardiomyocytes is observed, suggesting they potentiate the biological effect of Activin A.

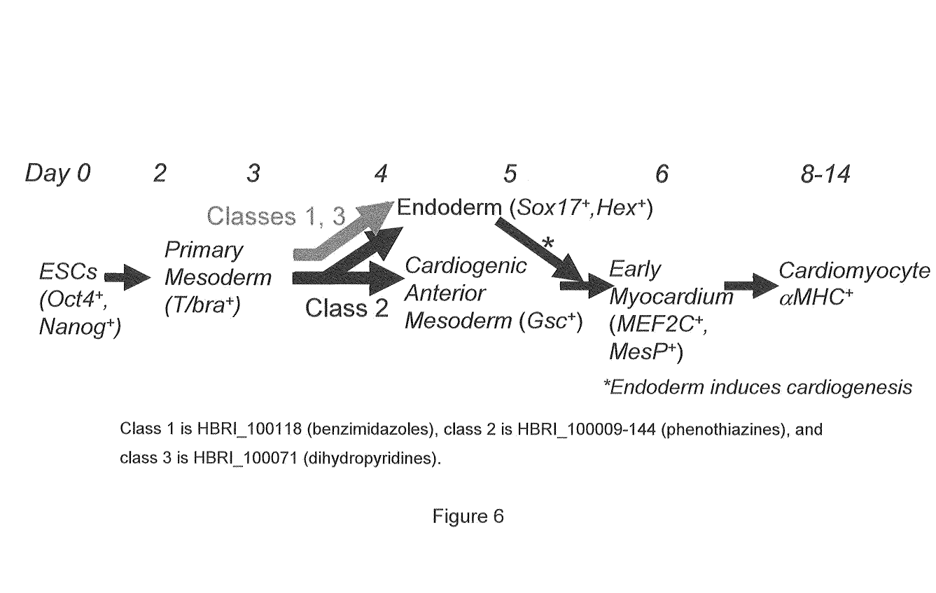

Using small molecule-treated day 4 EBs in small scale transcriptional profiling of typical mesoderm markers suggests that HBRI_{—}100071 a dihydropyridine) enhances the induction of anterior mesoderm marked by Gsc (mesoderm part) and Sox17 (endoderm part), whereas HBRI_{—}100118 a (benzimidazole) and HBRI_{—}100009-144 a phenothiazine) only promote the formation of endoderm and do not appear to affect anterior mesoderm. None of the small molecules were found to induce pan-mesoderm markers such as Brachyury or Flk1, indicating they are involved in mesoderm patterning rather than mesoderm induction. Based on marker analysis, the series including HBRI_{—}100071 dihydropyridines) would enhance cardiogenesis by enhancing anterior mesoderm formation, but might also stimulate cardiogenesis indirectly by promoting endoderm. In contrast, the marker analysis indicates that the series including HBRI_{—}100118 (benzimidazoles) and HBRI_{—}100009-144 (phenothiazines) act to induce endoderm only and thus would therefore stimulate cardiogenesis indirectly since the endoderm would provide the natural signals that direct primary mesoderm to become cardiac mesoderm. FIG. 6 summarizes the conclusions of the biological mechanism of action studies.

To begin to investigate signaling pathways targeted by the compounds, we asked if they mimic or synergize with Wnt signaling. Wnt signaling is known to synergize with Activin/Nodal signaling to induce and pattern mesoderm. To date, we have studied the HBRI_{—}100118 (benzimidazoles) and HBRI_{—}100071 (dihydropyridines) series.

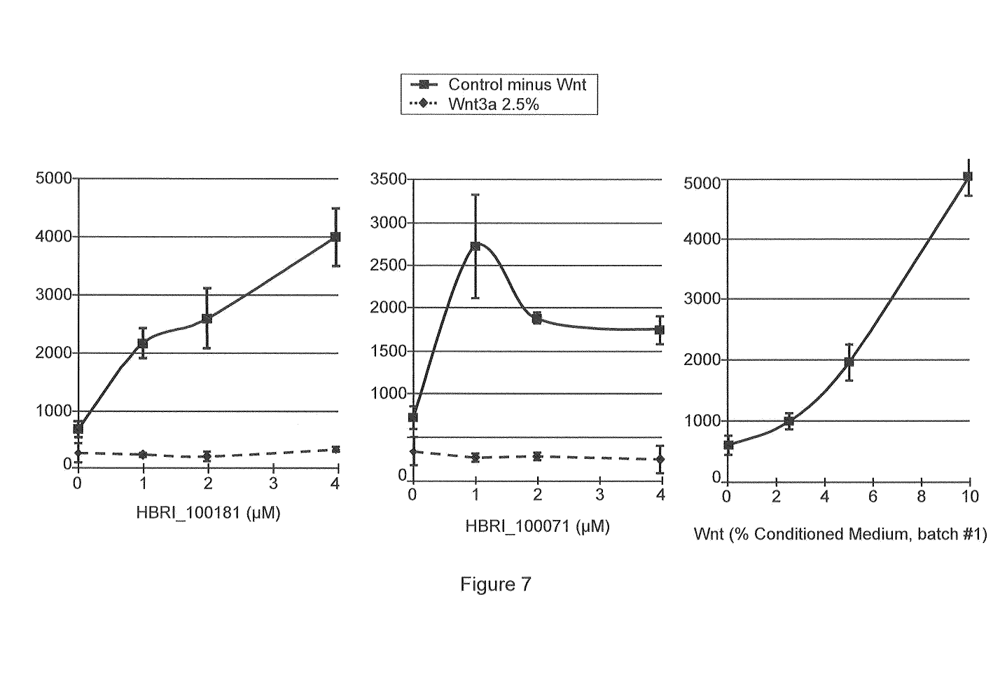

Signaling is tested using a standard luciferase response system for canonical Wnt/b-catenin/TCF signaling. Briefly, we used a cell line (RKO) that had been stably transfected with a luciferase reporter gene under the control of the response element for T CF₃, a transcription factor that is activated by association with beta-catenin and is the target of canonical Wnt signaling. Using this assay, HBRI_{—}—100118 and HBRI_{—}100071 did not activate luciferase; thus, they do not mimic Wnt/beta-catenin signaling. However, both increased activity of a submaximal dose of Wnt3a (FIG. 7), indicating that they activate signals that converge on the pathway to increase its activity.

The signaling effects of the phenothiazine series is under investigation. Also, we are investigating the effect of the compounds on Activin/Nodal signaling using an analogous luciferase expression system.

To summarize, using an assay based on a mouse ESC reporter line with GFP under control of the cardiac specific alpha Myosin heavy chain (aMHC) gene, 3 distinct chemical classes of molecules were identified, dihydropyridines, benzimidazoles and phenothiazines, as discussed above. They were found to act in the early window of mesoderm differentiation at the point of dictating mesoderm and endodermal lineages. Due to the fact that the original assay is performed in the presence of serum, which activates many pathways that obscure the targets of the small molecules, analysis of downstream targets is more difficult. To facilitate the analysis of these compounds, a serum-free mESC cardiac differentiation assay is developed that allows the study of growth factors and/or small molecules in the early mesoderm differentiation time frame and their effect on cardiogenesis. Results with this assay revealed that the hits “synergize” with Activin/Nodal signaling, but do not themselves activate this pathway.

In summary, each series of compounds appeared to promote cardiomyogenesis at a different time point in the differentiation cascade. Based on the initial “hit,” a dihydropyridine, approximately 100 analogs were synthesized that provided a drug-like “smart library” with various new chemical substituents. Some of the results are provided in some of the above Examples. The analogs were tested in the cardiomyocyte assay described above and the results showed a structure-activity relationship (SAR) for the “smart library”. Several of the synthetic analogs showed increased activity (i.e., IC₅₀ values in the 0.06 and 2.1 um range) and possessed greater drug-like properties.

For the second “hit,” a benzimidazole, over 600 analogs of that class were synthesized, tested in the cardiomyocyte assay and the data also described an SAR for this second drug-like “smart library.”

For the third “hit,” a Tamoxifen analog, over 100 analogs to add in view of comment mlg28. For the fourth “hit” (i.e., phenothiazine), a “smart library” of a total of 45 analogs were synthesized.

Small scale transcriptional profiling of typical mesoderm markers suggested that benzimidazoles enhance the induction of anterior mesoderm marked by Gsc (mesoderm part) and Sox 17 (endoderm part), whereas dihydropyridines only promote the formation of endoderm and does not appear to affect mesoderm. None of the small molecules were found to induce pan-mesoderm markers such as Brachyury or Flk1, indicating they are involved in mesoderm patterning rather than mesoderm induction. In conclusion, these classes of small molecules act by patterning uncommitted primary mesoderm into endoderm and cardiogenic mesoderm.

Although the disclosure has been described with reference to the above examples, it will be understood that modifications and variations are encompassed within the spirit and scope of the disclosure. Accordingly, the disclosure is limited only by the following claims.

1. A compound of Formula I: 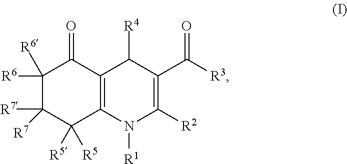
or a pharmaceutically acceptable salt or solvate thereof, wherein:
R¹ is independently hydrogen, (C₁-C₆)alkyl or a moiety forming a salt;
R² is independently hydrogen, (C₁-C₆)alkyl, CF₃ or C₂F₅;
R³ is independently OR^{S} or NR⁸R⁸;
R⁴ is independently substituted or unsubstituted phenyl, substituted or unsubstituted pyridine, wherein phenyl or pyridine is optionally independently substituted with 1 to 3 R⁹ substituents;
R⁵, R^{5′}, R⁶, R^{6′}, R⁷, and R^{7′} are each independently hydrogen or (C₁-C₆)alkyl;
R⁸ and R^{8′} are each independently hydrogen, (C₁-C₆)alkyl, (C₃-C₈)cycloalkyl, substituted
or unsubstituted heterocyclyl, aryl, (C₁-C₆)alkylaryl, or (C₁-C₆)alkylNR¹⁰R^{10′};
each R⁹ is independently hydrogen, halogen, (C₁-C₆)alkyl, (C₁-C₆)alkoxy, cyclo(C₁-C₆)alkyl, substituted or unsubstituted phenyl, substituted or unsubstituted pyridine, substituted or unsubstituted indolyl; substituted or unsubstituted pyrrolidinyl, or substituted or unsubstituted piperidinyl, wherein phenyl, pyridine, indolyl, pyrrolidinyl and piperidinyl are each optionally independently substituted with hydrogen, halogen, or (C₁-C₆)alkyl; and
R¹⁰ and R^{10′} are each independently hydrogen, (C₁-C₆)alkyl, aryl, or (C₁-C₆)alkylaryl.

2. The compound of claim 1, wherein R¹ is hydrogen; R² is hydrogen, CH₃ or CH₂CH₃; R³ is OR⁸; R⁴ is substituted or unsubstituted phenyl; R⁸ is hydrogen, CH₃, CH₂CH₃, 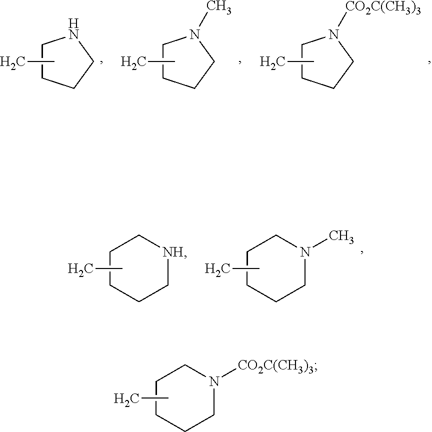
and each R⁹ is independently hydrogen, F, Cl, Br, or I.

3. The compound of claim 2, wherein R⁸ is CH₃, CH₂CH₃,

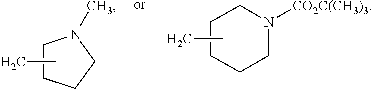

4. The compound of claim 1, wherein the compound of Formula I has Formula IC: 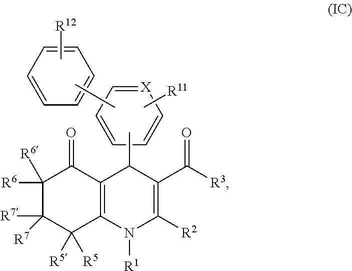
or a pharmaceutically acceptable salt or solvate thereof, wherein X is CH or N; and R¹¹ and R¹² are each independently hydrogen, halogen, or (C₁-C₆)alkyl.

5. The compound of claim 4, wherein X is CH; R¹ is hydrogen; R² is hydrogen, CH₃ or CH₂CH₃; R³ is OR^{B}; R⁸ is CH₃, CH₂CH₃, 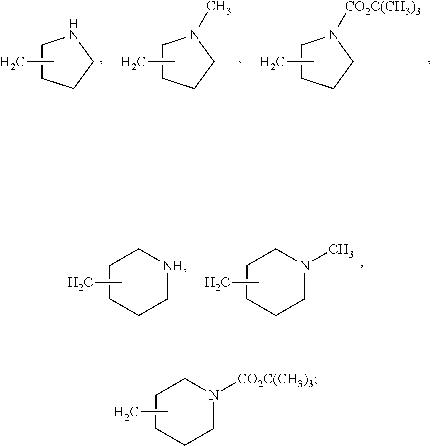
and R¹¹ and R¹² are each independently hydrogen, F, Cl, Br, I, CH₃ or CH₂CH₃.

6. The compound of claim 5, wherein R⁸ is CH₃, CH₂CH₃,

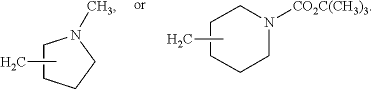

7. The compound of claim 1, wherein the compound of Formula I has Formula ID: 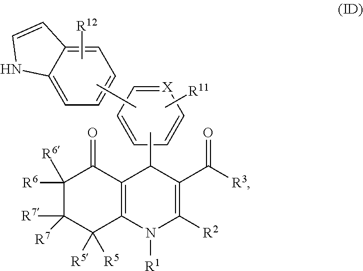
or a pharmaceutically acceptable salt thereof, wherein X is CH or N; and R¹¹ and R¹² are each independently hydrogen, halogen, or (C₁-C₆)alkyl.

8. The compound of claim 7, wherein R¹ is hydrogen; R² is hydrogen, CH₃ or CH₂CH₃; R³ is OR⁸; R⁸ is CH₃, CH₂CH₃, 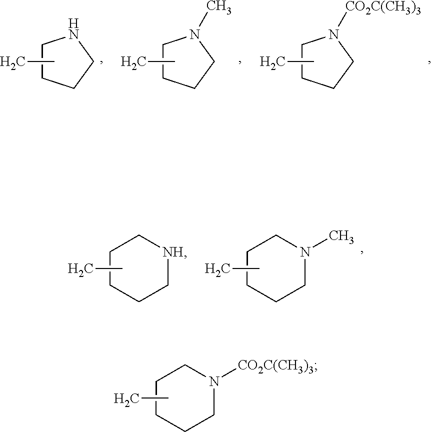
and R¹¹ and R¹² are each independently hydrogen, F, Cl, Br, I, CH₃ or CH₂CH₃.

9. The compound of claim 8, wherein R⁸ is CH₃, CH₂CH₃,

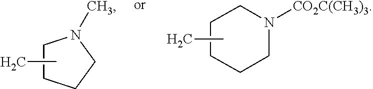

10. The compound of Formula I of claim 1, having formula:

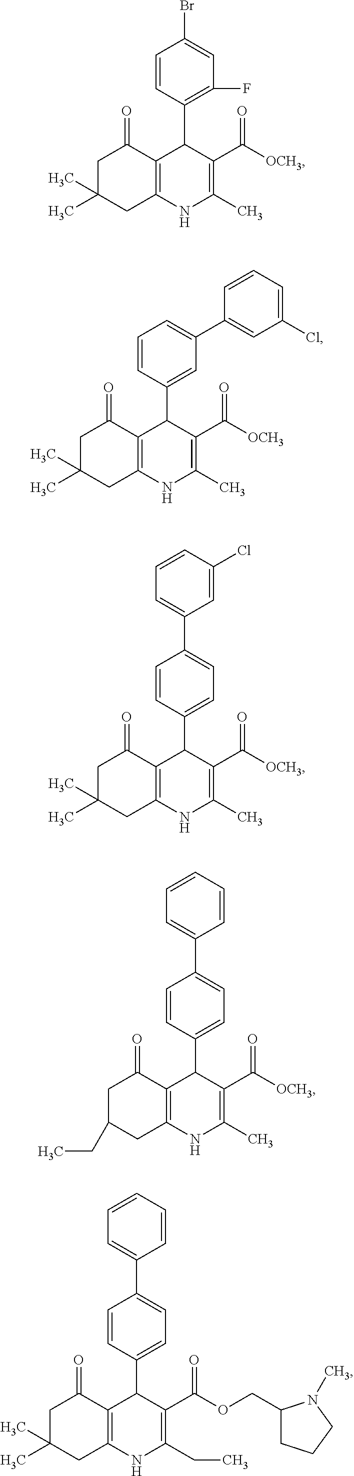

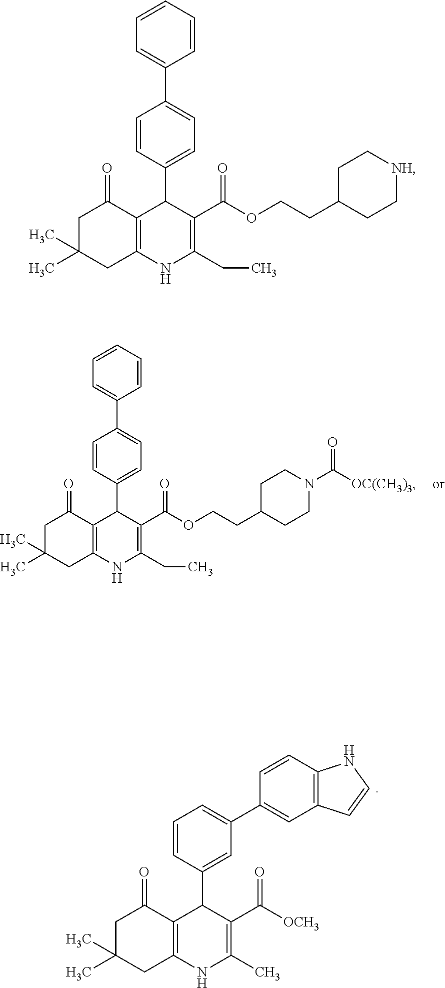

11. The compound of claim 1, wherein the pharmaceutically acceptable salt is the salt of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), or undecylenic acid.

12. A method for producing differentiated cells from stem cells, comprising contacting the stem cells with a compound of claim 1 of Formula I.

13. The method of claim 12, wherein contacting is from about 24 hours to about 192 hours.

14. The method of claim 12, wherein contacting is from about 48 hours to about 144 hours.

15. The method of claim 12, wherein the differentiated cells are cardiomyocytes, hepatocytes, or islet cells.

16. The method of claim 12, further comprising contacting the cells with Activin A.

17. The method of claim 12, wherein the cells differentiate to mesoderm.

18. The method of claim 12, further comprising contacting the cells with a Wnt protein.

19. The method of claim 18, wherein the Wnt protein is Wnt3a, 5a or 7.

20. The method of claim 12, wherein the stem cells are embryonic stem cells, induced pluripotent stem cells or adult stem cells.
